(19) United States
(12) Patent Application Publication
(10) Pub. No.: US 2003/0207391 A1
(43) Pub. Date: Nov. 6, 2003

(54) Binding protein

(76) Inventor: Helen Pappa, Wokingham (GB)

Correspondence Address: KLAUBER & JACKSON, 411 HACKENSACK AVENUE, HACKENSACK, NJ, 07601

(21) Appl. No.: 10/165,193

(22) Filed: Jun. 7, 2002

Related U.S. Application Data
(63) Continuation of application No. PCT/GB00/04693, filed on Dec. 7, 2000.

(30) Foreign Application Priority Data
Dec. 7, 1999 (GB) 9928950.6

(51) Int. Cl. C12P 21/02; C12N 5/06; C07H 21/04; C07K 14/71

(52) U.S. Cl. 435/69.1; 435/320.1; 435/325; 530/350; 536/23.5

(57) ABSTRACT

This invention relates to novel compounds that act to prevent dimerisation of vascular endothelial growth factor (VEGF) receptors. The novel compounds may comprise the amino acid sequence of the fourth Ig-like domain of a VEGF receptor, or a variant that retains the ability to bind to a VEGF receptor. These compounds are useful in the inhibition of the biological activity of VEGF receptors and may thus be used to treat diseases in which VEGF plays a role.

The present invention relates to novel proteins that act to prevent dimerisation of vascular endothelial growth factor (VEGF) receptors. These proteins are useful in the inhibition of the biological activity of VEGF receptors and may thus be used to treat diseases in which VEGF plays a role.

VEGF is a potent stimulator of angiogenesis and plays an important role in the mammalian body in the development of the vascular system. It has been implicated in various human diseases such as inflammation, psoriasis, rheumatoid arthritis, hemangiomas, diabetic retinopathy, angiofibromas, macular degeneration, endometriosis, retinal neovascularisation and cancer. The molecule has been implicated particularly in solid tumours, whose growth can be prevented by the inhibition of VEGF action (Kim et al., (1993) Nature 362: 841-844)

VEGF plays a role in endometriosis (McLaren et al., (1996) Human Reproduction 11, No. 1, 220-223; McLaren et al., (1996) J. Clin. Invest. 98 No. 2, 482-489), the name given to the disease that results from the presence of endometrium outside the uterine cavity. This disease affects women during their childbearing years with deleterious social, sexual and reproductive consequences. Endometriosis has been proposed as one of the most commonly-encountered diseases of gynaecology, with the incidence of endometriosis in the general population being estimated to be around 5%, although it is thought that at least 25% of women in their thirties and forties may be suffering from this disease.

The VEGF family consists of VEGF-A, VEGF-B, VEGF-C, VEGF-D, VEGF-E, and PLGF as well as their spliced variants. The biological activity of the VEGF family is mediated in vivo by three receptor tyrosine kinases that are primarily expressed in endothelial cells: kinase insert domain-containing receptor (KDR/FLK-1); the FMS-like tyrosine kinase receptor-1 (FLT-1); and the FMS-like tyrosine kinase receptor-4 (FLT-4) which exhibit high binding affinity for the VEGF family members (de Vries et al., (1992) Science 255: 989-991; Terman et al., (1992) Biochem. Biophys. Res. Commun. 187; 1579-1586; Davis-Smyth et al., (1996) EMBO Journal 15: 4919-4927). Additionally, the VEGF-A 165 isoform binds to neuropilin-1 (Soker S et al. (1998) Cell 92(6): 735-745) and neuropilin-2 (Neufeld G et al. (1999) FASEB J., 13(1): 9-22).

All of FLT-1, KDR/FLK-1 and FLT-4 are membrane-spanning receptors with an extracellular ligand-binding region containing seven immunoglobulin-like domains, a transmembrane domain and an intracellular tyrosine kinase domain. The transmembrane domain serves to anchor the receptor in the membrane of the cells in which it is expressed. The biological activities of the FLT-1 and KDR receptors have been shown to differ, implying that these proteins have different functions in vivo (Roeckl et al., (1998) Experimental Cell Research 241: 161-170; Shalaby et al., (1995) Nature 376: 62-66; Fong G et al., (1995) Nature 376: 66-70). FLT-4 is not a receptor for VEGF-A but rather binds to VEGF-C and VEGF-related protein (VRP). Like VEGF, VEGF-C and VRP can induce mitogenesis in vascular endothelial cells, but at 100-fold less potency (Lee J et al., (1996) Proc. Natl. Acad. Sci. USA 93: 1988-1992).

Various groups have investigated the structure of VEGF receptors in relation to the biological functions of these molecules. Keyt et al., (1996) (J. Biol. Chem. 271 (10) -5638-5646) mapped the residues important for VEGF binding on both KDR and FLT-1 and suggested that VEGF displays different receptor binding sites for KDR and FLT-1.

Shinkai et al., (1998) (Journal of Biological Chemistry 273 (47): 31283-31288) mapped various sites of the extracellular domain of the KDR receptor thought to be involved in ligand association and disassociation and concluded that the third Ig-like domain is critical for ligand binding with the second and fourth domains playing a role in ligand association. The fifth and sixth domains are required for retention of the ligand when bound to the receptor molecule, while the first Ig-like domain was proposed to regulate ligand binding.

Davis-Smyth et al., (1996) (EMBO Journal 15 (18): 4919-4927) reported that the second Ig-like domain of the FLT-1 receptor contains critical determinants of ligand binding. Furthermore, when the FLT-4 domain 2 was exchanged for that of FLT-1, FLT-4 became non-responsive to its natural ligand VEGF-C, suggesting that domain 2 is also critical for binding in the FLT-4 receptor. These findings suggested that determinants for binding and ligand specificity within the second Ig-like domain is a common feature of subclass III receptor tyrosine kinases with seven Ig-like domains. Later studies proved that domains 2 and 3 are necessary for ligand binding with wild type affinity (Barleon et al., (1997) J. Biol. Chem. 272 (16): 10382-10388; Davis-Smyth et al., (1998) 273 (6): 3216-3222; Wiesmann et al., (1997), Cell 91: 695-704). The ligand-FLT-1 domain 2 interactions were determined in detail by the determination of the high resolution structure of FLT-1 domain 2 with VEGF (Wiesmann et al., 1997, Cell 91; 695-704).

In an independent study, Barleon et al. (1997) (J. Biol. Chem. 272 (16); 10382-10388) mapped the sites for ligand binding and receptor dimerisation in the extracellular domain of the FLT-1 receptor and confirmed that the first three Ig-like loops are involved in high affinity binding of VEGF. Dimerisation of the extracellular domains of FLT-1 receptor was only detected in the constructs that contain the fourth Ig-like loop.

Kendall & Thomas, 1993 (P.N.A.S. USA 90; 10705-10709) reported the cloning of a soluble truncated form of FLT-1 from a human vascular endothelial cell library. This molecule was found to comprise the six N terminal immunoglobulin-like extracellular ligand-binding domains but to lack the transmembrane-spanning region and intracellular tyrosine kinase domains. Binding affinity for VEGF-A was retained, prompting these workers to speculate that this soluble receptor might act as an efficient specific antagonist of VEGF in vivo.

Clark et al., (1998) (Biol. Reprod. 59: 1540-1548) reported the occurrence of soluble FLT-1 (sFLT-1) in serum from pregnant women, which was not present in serum from men and from non-pregnant women. These workers thus speculated the in vivo production of the FLT-1 receptor might constitute a mechanism for naturally-regulating VEGF-induced angiogenesis. No naturally-occurring secreted form of KDR has been reported to date, however, sFLT-1 has been shown to form ligand-induced heterodimeric complexes with full length KDR (Kendall et al., (1996), Biochem. Biophys. Res. Commun. 226 (2): 324-328). To the best of the Applicant's, there is no naturally-occurring secreted form of FLT-4.

Certain approaches have been suggested that attempt to treat VEGF mediated disease by supplying VEGF antagonists such as neutralising antibodies, VEGF receptor molecules, and portions of such receptor molecules (see, for example co-pending patent application PCT/GB95/01213, Metris Therapeutics; PCT/US97/17044, Merck & Co., Inc.; PCT/US97/07694, Genentech, Inc.; PCT/US92/09218, Genentech, Inc.; PCT/US94/01957, Merck & Co., Inc.). However, all of the approaches embodied in these patent applications rely on reducing the effective concentration of VEGF molecules, and none of the suggested approaches have yet provided agents that are effective against all types of VEGF-mediated disease.

There thus remains a great need for novel compounds that are effective to disrupt VEGF function in vivo.

### SUMMARY OF THE INVENTION

According to a first aspect of the invention there is provided a protein consisting of the amino acid sequence of the fourth Ig-like domain of a VEGF receptor, a variant of said protein that retains the ability to bind to a VEGF receptor or a functional equivalent of said fourth Ig-like domain.

All VEGF receptors form homodimers. The VEGF molecule itself acts as a dimer, and the binding of one monomer component to a receptor molecule induces the dimerisation of the VEGF receptor molecule through the interaction of the second monomer component with a second VEGF receptor in the cell membrane (Fuh et al., 1998, J. Biol. Chem. 273, No. 18, 11197-11204). The dimerisation of the VEGF receptor molecule induces the activation of the intracellular kinase domain of the receptor, thus initiating the signal transduction cascade that is effective to translate the ligand-receptor binding event into the activation of the appropriate secondary messenger system in the cell.

The present invention provides molecules that when bound to the fourth Ig-like domain of a full length VEGF receptor, prevent its dimerisation. Since VEGF-dependent activation of the intracellular signalling domain of the full-length receptor occurs only when the VEGF receptor is in its dimeric state, blocking the dimerisation event severs the link between ligand binding and activation of the secondary messenger system. Accordingly, the biological action of VEGF may be specifically blocked.

The invention has a number of advantages over systems that have been previously described. Most of these systems involve mechanisms that are designed to remove the effective amount of VEGF from circulation, either systemically, or in specific areas of the body. Such techniques are far from ideal for a number of reasons, the most obvious being that VEGF is a molecule with a wide range of biological functions in the body. Lowering the effective levels of this molecule, either by preventing its expression, or by interfering with it directly through specific binding events, acts to abolish VEGF function altogether. so leading to unwanted side-effects. Studies that have used small molecule inhibitors to target VEGF-Receptor tyrosine kinases may inhibit other kinases and so cause unwanted side-effects.

One advantage of using the molecules of the present invention in therapy is that VEGF itself is left unaffected, so a free population of the VEGF molecule remains to perform its natural biological functions. By targeting the receptor molecules themselves, VEGF levels remain unchanged, meaning that the normal VEGF-mediated processes are allowed to continue unaltered by the therapy process. Furthermore, the molecules of the present invention may be designed to target only a subset of VEGF receptor types, so leaving non-targeted receptors unaffected, meaning that this method of therapy is unlikely to cause undesirable side-effects.

By the term the “fourth Ig-like domain” is meant the immunoglobulin-like domain of the VEGF receptor that is considered by the inventors to be necessary for the dimerisation function of the receptor molecule. This domain is defined as being the fourth Ig-like domain as counted from the NH₂ terminus of the receptor molecule. It is not at present clear which precise residues participate in the dimerisation event. However, the molecules of the invention should retain sufficient residues from the fourth Ig-like domain to bind to the corresponding domain of a VEGF receptor with high enough affinity to compete effectively for binding with wild type full length VEGF receptor molecules.

At present, there are three VEGF receptors known. However, the present invention is likely to be equally applicable to other VEGF receptors that are discovered in the future. Preferably, the proteins of the present invention are derived from the receptors FLT-1, FLK/KDR and FLT-4. The VEGF receptors to which the proteins of the invention bind are preferably mammalian, most preferably human VEGF receptors.

In FLT-1, the boundaries of the fourth Ig-like domain are considered to be within amino acid residues 316 or 317 and 447 inclusive, wherein the numbering system starts at the first methionine residue in FIG. 1. However, shorter protein molecules may be used, provided that the molecules include at least amino acid residues 344-406 of the FLT-1 sequence. Preferably, those proteins of the invention that are derived from FLT-1 consist of at least residues 338-440, more preferably 330-440 of the full length FLT-1 sequence. Examples of particularly preferred constructs include those that consist of residues 330-440, 330-429 or 338-429 of the full length FLT-1 sequence.

The analogous residues in the FLK receptor are shown in FIG. 5. The boundaries of the fourth Ig-like domain of this receptor is considered to be at residues 314 and 440 of the full length FLK sequence given in FIG. 2. At the very least, this domain should include residues 342-404 of the FLK sequence. Preferably, those proteins of the invention that are derived from FLK consist of at residues 336-439, 335-435, 335-424, more preferably residues 328-424 or 328-435 of the full length FLK sequence.

The analogous residues in the KDR receptor are also shown in FIG. 5. The boundaries of the fourth Ig-like domain of this receptor are considered to be at residues 312-438 of the full length sequence given in FIG. 3. At the very least, this domain should include residues 340 to 402 of the KDR sequence. Preferably, those proteins of the invention that are derived from KDR consist of at residues 333-438, 333-433 or 333-422, more preferably residues 326-422 or 326-433 of the full length KDR sequence.

The analogous residues in the FLT-4 receptor are also shown in FIG. 5. The boundaries of the fourth Ig-like domain of this receptor are considered to be residues 315 to 437 of the full length sequence given in FIG. 4. At the very least, this domain should include residues' 343-403 of the FLT-4 sequence. Preferably, those proteins of the invention that are derived from FLT-4 consist of residues 339-437, 339-423, more preferably, 329-423 or 329-437 of the full length FLT-4 sequence.

The boundaries of the fourth Ig-like domain were predicted, based on sequence alignments of FLT-1 and KDR with telokin, as well as secondary structure predictions assisted by the crystal structure of the telokin molecule (PDB file: ITLK).

The maximum boundaries of the fourth Ig-like domain were determined to reside within a few residues of the last conserved cysteine residue in the third Ig-like domain, and the first conserved cysteine residue of the fifth Ig-like domain (Cys311 and Cys454 respectively).

The minimum boundaries were defined after aligning FLT-1 to telokin and defining the minimum number of secondary structure elements that would be sufficient to support a stable protein fold.

Additionally, information of the exon-intron genomic organisation of FLT-1 was generated by the Applicant, constructed from sequencing data generated from the human genome sequencing effort. FLT-1 resides within chromosome 13; the genomic organisation of this gene was deciphered using recently deposited sequencing data of chromosome 13 clones.

The identification of the appropriate boundaries of other VEGF receptors to which the teaching of the present invention may be applied will be clear to those of skill in the art. Details of the boundaries of other preferred constructs are shown in Table 1.

The proteins of the present invention may comprise an amino acid sequence that corresponds exactly to the wild type receptor protein sequence found in the fourth Ig-like domain of the VEGF receptor protein. The wild type amino acid sequences of the fourth Ig-like domains of the FLT-1, FLK/KDR and FLT-4 receptors are shown in FIG. 5. However, as the skilled reader will appreciate, the proteins of the invention may be derived from any mammalian VEGF receptor sequence. Human sequences are preferred.

As used herein, the term “wild type” means the amino acid sequence that is characteristic of most of the members of the particular species from which the receptor molecule is derived. Included within the term “wild type” are natural biological variants of the VEGF receptor molecule sequences (for example, allelic variants or geographical variations within the species from which the wild type proteins are derived).

The proteins of the present invention may most suitably be derived from VEGF receptors in the FLT-1, KDR/FLK and FLT-4 receptor family. All of these molecules dimerise through the interaction of the respective fourth Ig-like domains of the molecule. Of particular applicability to the present invention are recombinant proteins derived from the FLT-1 and KDR/FLK proteins. These molecules, and the effect of these molecules when aberrantly-regulated or when mutated, is thought to have a particularly important role in the pathology of diseases such as cancer and endometriosis.

Variants of wild type fourth Ig-like domain receptor sequences are also included in the present invention. As the skilled man will appreciate, the term “variant” includes molecules that contain single or multiple amino-acid substitution(s), addition(s), insertion(s) and/or deletion(s) from the wild type protein sequence, provided that such variants maintain the ability to bind to the corresponding fourth Ig-like domain of target VEGF receptor and thus prevent their dimerisation. Variant molecules may also contain substitutions of chemically-modified or synthetic amino acids that do not affect the function or activity of the protein in an adverse manner.

Suitable variants of the molecules of the invention will be those proteins that exhibit high affinity for the fourth Ig-like domain of a VEGF receptor. Preferably, this affinity is higher than that of the wild type sequence. Typically, the protein according to the present invention binds to a VEGF receptor with a dissociation constant of 2 μM or less, preferably, 0.2 μM or less, more preferably 2 nM or less, even more preferably, 20 pm or less.

Another property that is desirable for a variant of the wild type sequence is the ability to bind to the fourth Ig-like domain of an intact VEGF receptor with a significantly higher affinity than the wild type protein displays for binding to the intact VEGF receptor. The dimerisation kinetics will lead towards the formation of heterodimers with target receptor with increasing concentrations of the proteins of the invention. Such concentrations will vary for different cell types as they will depend on the number of target receptor molecules that are present on the cell surface. However, a large excess of protein should be able to mask all cell surface receptors, so conferring a therapeutic effect to the patients to whom preparations of these proteins will be administered.

The term “functional equivalent” is used herein to describe proteins that have an analogous function to the fourth Ig-like domain of a VEGF receptor and that bind specifically to this domain, thus preventing dimerisation of the receptor molecules and so inhibiting signal transduction effected by ligand binding to the VEGF receptor. This term therefore includes molecules that are structurally similar to the fourth Ig-like domains identified herein or that contain a similar or identical tertiary structure. The analogous binding properties of functional equivalents should be reflected in their affinity for the fourth Iglike domain of a VEGF receptor. Typically, functional equivalents should bind to a VEGF receptor with a dissociation constant of 2 μM or less, preferably, 0.2 μM or less, more preferably 2 nM or less, even more preferably, 20 pm or less. For these molecules, the thermodynamics of binding should be sufficient that physiologically-attainable concentrations of molecule are effective to prevent VEGF receptor dimerisation.

The term “functional equivalent” therefore includes entities such as antibodies, (particularly antiidiotypic antibodies), oligopeptides, peptides, peptidomimetics, drug molecules such as small natural or synthetic organic molecules of up to 2000Da, preferably 800Da or less in size. Other examples of functional equivalent molecules will be clear to those of skill in the art.

Functionally-equivalent peptide, oligopeptide or polypeptide compounds according to the present invention may be generated by any suitable means, as will be apparent to those of skill in the art. In addition to the naturally-occurring amino acids, these molecules may, of course, contain synthetic amino acids.

In the case of antiidiotypic antibodies, these may be obtained by immunisation of an appropriate host with a preparation of an antibody that recognises the dimerisation interface within the fourth Ig-like domain molecule.

In the case of peptides, combinatorial peptide libraries may be most suitable to isolate peptide molecules that display the desired binding characteristics, through the use of selection regimes that select for molecules that bind to antibodies that are specific for the fourth Ig-like domain of a VEGF receptor.

One method of generation of peptide libraries utilises degenerate oligonucleotide libraries. This method allows the subsequent analysis of the encoding nucleic acid and thus gives direct sequence information for the mimotope (see for example, Cull et al. (1992); Matteakis et al., (1994)).

Phage display technology also provides a vehicle that allows for the selection of displayed peptides, oligopeptides or polypeptides and that simultaneously provides a link between phenotype and genotype so that the encoding nucleic acid can be identified and analysed (for a review see Clackson and Wells (1994) Trends Biotechnol 12: 173-184). Filamentous phage particles act as genetic display packages with proteins on the outside and the nucleic acids that encode them on the inside. The practical limit on library size allowed by this technology is of the order of 10⁷ to 10¹¹ variants, so allowing the generation of a huge number of different compounds. This technology also allows iterative rounds of selection to be performed, so honing the affinity of the molecules isolated.

The preferred method of generation of peptide, oligopeptide or polypeptide compounds that are functional equivalents of the proteins of the invention is through selection of candidate compounds in a phage display library.

Selection of a nucleic acid or gene from a phage display library will in most cases require the screening of a large number of variant nucleic acids or genes. Libraries of nucleic acids or genes for use with phage display technology may be generated in a variety of ways. For example, pools of naturally-occurring genes may be cloned from genomic DNA or cDNA (see Sambrook et al., 1989). Phage-antibody libraries, made by PCR amplification repertoires of antibody genes from immunised or non-immunised donors have proved very effective sources of functional antibody fragments (Winter et al., (1994) Annu Rev Immunol, 12: 433-55; Hoogenboom, (1997) Trends Biotechnol. 15: 62-70).

Libraries of genes can also be made by encoding all or part of genes or pools of genes or by using randomised or doped synthetic oligonucleotides. Libraries can also be made by randomly introducing mutations into a gene or into a pool of genes by a variety of techniques in vivo, including using so-called ‘mutator strains’ of bacteria such as E. coli mutD5 (Liao et al., (1986) P.N.A.S. USA, 83: 576-580).

Random mutations can also be introduced both in vivo and in vitro by chemical mutagens, and ionizing or UV irradiation (see Friedberg et al., (1995) DNA repair and mutagenesis. ASM Press, Washington), or by incorporation of mutagenic base analogues (Zaccolo et al., (1996) J Mol Biol 255: 589-603). Mutations can also be introduced into genes in vitro during polymerisation, for example by using error-prone polymerases (Leung et al., (1989) Technique, 1: 11-15). Further diversification can be introduced by using homologous recombination either in vivo (see Kowalczykowski et al., (1994) Microbiol Rev, 58: 401-465) or in vitro. (Stemmer, (1994) Nature, 0.370: 389-391). Alternatively, directed mutagenesis may be performed according to methods well known in the art (see McPherson et al., (1991) Directed mutagenesis. A Practical Approach. IRL Press, Oxford).

The proteins may be specific for a class of VEGF receptors for example, the proteins may bind with high affinity to both the FLT-1, KDR/FLK and FLT-4 VEGF receptors, thus abolishing VEGF-mediated activity via all these receptor types.

In an alternative embodiment, the proteins of the invention may be specific for one particular type of receptor, for example, FLT-1. In this manner, specific VEGF pathways can be targeted, that are responsible for a particular disease, or for a particular aspect of a disease. In this manner, the risk of toxic side-effects can be reduced.

In a further embodiment, the proteins of the invention may comprise hybrid molecules consisting of multiple components. Such proteins of the invention may consist of repeated amino acid sequences of the fourth Ig-like domain of one or more VEGF receptors. Proteins of this aspect of the invention may dimerise more efficiently to VEGF receptor target molecules through possessing multiple binding sites.

In an alternative embodiment, such repeated domains may be derived from different receptors. For example, a protein may comprise an amino acid sequence derived partially from the fourth Ig-like domain of the FLT-1 receptor and partially from the fourth Ig-like domain of the KDR/FLK or FLT-4 receptor. In this manner, proteins according to the invention may be designed rationally so as to impart specific binding properties of interest, such as increased affinity for a certain receptor molecule. The protein may thus be designed so as to interfere with a specific dimerisation event, for example, the dimerisation of the FLT-1 receptor. The normal dimerisation process exhibited by other VEGF receptors, for example a KDR receptor, may therefore be unaffected.

According to a further aspect of the invention there is provided a protein or functional equivalent according to any one of the above-described aspects of the invention that has been genetically or chemically fused to one or more peptides or polypeptides. For example, dimerisation of the proteins of the invention with each other might be prevented by fusing the fourth Ig-like domain to an effector domain that acts in the absence of ligand to prevent homodimerisation of the protein. On binding of a ligand to the effector domain, a change in the conformation of the fusion protein would allow dissociation of the fourth Ig-like domain portion of the protein, leaving it free to bind to its target VEGF receptor molecule in situ.

Other components suitable for fusion with proteins of the invention include labels, such as a radioactive, enzymatic, fluorescent, or antibody label. In this embodiment of the invention, fusion proteins can be used as diagnostic tools in the evaluation of the disease state of a patient. Other suitable components for fusion include bioactive moieties such as toxins that could be delivered to specific cell types.

The proteins of the invention are preferably recombinant, meaning that they are derived by recombinant DNA technology. Recombinant expression of proteins allows a high level of expression to be obtained at an economic cost. Recombinant expression is widely known in the art and involves the incorporation of the gene encoding the protein of interest into an expression vector. Such an expression vector will incorporate appropriate transcriptional and translational control sequences, for example enhancer elements, promoter-operator regions, termination stop sequences, mRNA stability sequences, start and stop codons or ribosomal binding sites, linked in-frame with the gene encoding the protein of interest. Secretion signalling and processing sequences may also be appropriate. Many suitable vectors and expression systems are well known and documented in the art (see, for example, Sambrook et al., Molecular Cloning: a laboratory manual; Cold Spring Harbor Laboratory Press; Fernandez & Hoeffler, 1998). Particularly suitable viral vectors include baculovirus-, adenovirus- and vaccinia virus-based vectors.

The proteins of the invention may be expressed recombinantly in prokaryotic hosts, such as in E. coli, or in eukaryotic yeasts that can be made to express high levels of recombinant protein and that can be grown easily in large quantities. Mammalian cell lines grown in vitro are also suitable, particularly when using virus-driven expression systems. Another suitable expression system is the baculovirus expression system that involves the use of insect cells as hosts. An expression system may also constitute host cells that have the encoding DNA incorporated into their genome. Recombinant protein may easily be purified from these host cells in large quantities and at an economic cost.

Proteins for the treatment of diseases such as cancer or endometriosis will generally be administered to patients as pharmaceutical compositions in therapeutically-effective doses. The term “therapeutically effective dose” as used herein refers to an amount of a therapeutic agent that is effective to treat, ameliorate, or prevent the disease in question, or to exhibit a detectable therapeutic or preventative effect. The precise effective amount for a subject will depend upon the subject's size and health, the nature and extent of the disease condition, and the therapeutic agent or combination of therapeutic agents selected for administration. The effective dose for each given situation can be determined by routine experimentation and is within the judgement of the skilled person. For example, in order to formulate a range of dosage values, cell culture assays and animal studies can be used.

The dosage of such compounds preferably lies within the dose that is therapeutically effective, and that exhibits little or no toxicity at this level. For example, an effective parenteral dose will be between 0.01 mg/ kg and 50 mg/kg or, more typically, between 0.05 mg/kg and 10 mg/kg of the individual to which it is administered.

According to a further aspect of the invention there is provided a protein according to any one of the above-described aspects of the invention, for use as a pharmaceutical.

The invention also provides the use of a protein according to any one of the above-described aspects of the invention in the manufacture of a medicament for the treatment of cancer, endometriosis, inflammation, psoriasis, rheumatoid arthritis, hemangiomas, diabetic retinopathy, angiofibromas, macular degeneration, retinal neovascularisation or any other disorder whose pathology is dependent upon a VEGF family-mediated pathway. Specific neoplasms and neoplastic conditions that are amenable to treatment include breast carcinomas, lung carcinomas, gastric carcinomas, oesophageal carcinomas, colorectal carcinomas, liver carcinomas, ovarian carcinomas, thecomas, arrhenoblastomas, cervical carcinomas, endometrial carcinomas, endometrial hyperplasias, fibrosarcomas, choriocarcinomas, head and neck cancers, nasopharyngeal carcinomas, hemangiomas, laryngeal carcinomas, hepatoblastomas, Kaposi's sarcomas, melanomas, skin carcinomas, cavernous hemangiomas, hemangioblastomas, pancreas carcinomas, retinoblastomas, astrocytomas, glioblastomas, Schwannomas, oligodendrogliomas, medulloblastomas, neurblastomas, rhabdomyosarcomas, osteogenic sarcomas, leiomyosarcomas, urinary tract carcinomas, thyroid carcinomas, Wilm's tumour, renal cell carcinomas, prostate carcinomas, abnormal vascular proliferation associated with phakomatoses, oedemas (such as that associated with brain tumours) and Meig's syndrome.

Specific non-neoplastic conditions that are amenable to treatment include rheumatoid arthritis, psoriasis, atherosclerosis, diabetic and other retinopathies, retrolental fibroplasias, leiomyomas, neovascular glaucomas, thyroid hyperplasias (including Grave's disease), corneal and other tissue transplantation, chronic inflammation, lung inflammation, nephrotic syndrome, pre-eclampsia, ascites, pericardial effusion (such as that associated with pericarditis) and pleural effusion.

The binding of proteins of the invention, or functional equivalents thereof, to the full length VEGF receptor or to truncated forms thereof can be used in high throughput screens for the identification of small molecular weight drug substances, such as small natural or synthetic organic molecules of up to 2000 Da, preferably 800 Da or less in size that are effective in inhibiting dimerisation of a VEGF receptor. These compounds may be peptidic or non-peptidic in nature. The assays can be radioactive, fluorescent, colorimetric, enzymatic, chemiluminescent or any other assay type that allows the quantitation of bound substance. In an alternative to the use of binding assays, enzyme assays such as tyrosine kinase assays may be used to assess the degree to which signal transduction is inhibited.

According to a still further aspect of the invention there is provided a nucleic acid encoding a protein according to any one of the above-described aspects of the invention. Such nucleic acid molecules may be incorporated into a suitable vector, which itself may be used to transfect a suitable host cell.

Nucleic acid molecules according to this aspect of the invention may in one aspect be used in the recombinant expression of the proteins of the above-described aspects of the invention.

In another aspect, such nucleic acid molecules may be used in gene therapy, to effect expression of the protein in situ. Gene therapy vehicles may comprise non-viral agents such as liposome formulations or may comprise recombinant viral vectors. Suitable viral vectors include, for example, vectors derived from retroviruses, adenoviruses, adeno-associated viruses, herpes viruses or papilloma viruses. Non-viral vectors include simple plasmids formulated, for example, as liposomes (Templeton and Lasic, (1999) Mol Biotechnol 11(2):175-80). Expression of the coding sequence can be induced using endogenous mammalian or heterologous promoters, and may be either constitutive or regulated. Suitable techniques for the introduction of gene therapy vehicles into cells include electroporation, the use of DNA guns, the direct injection of pure nucleic acid into tissue and liposome-mediated techniques (Dachs et al., (1997) Oncol. Res. 9(6-7): 313-325; Templeton and Lasic, (1999) Mol Biotechnol 11(2): 175-80). Gene therapy vehicles may be administered either locally or systemically.

An alternative form of gene therapy involves the introduction of cells, preferably autologous host cells, that contain nucleic acid sequences according to the above-described aspects of the invention into a patient suffering from a VEGF-mediated disease or disorder. The cells may comprise autologous cells harvested from the patient and transfected ex vivo with one or more nucleic acids encoding proteins according to the above-described aspects of the invention. These cells may then be transplanted back into the patient in areas that allow for the amelioration of disease symptoms to restore the healthy function of the tissue and prevent disease progression (Bailey C. J. et al., (1999) J. Mol. Med. 77(1): 244-249; Falqui L. et al., (1999) Hum. Gene. Ther. 10(11): 1753-1762).

According to a further aspect of the invention there is provided a pharmaceutical composition comprising a protein or a nucleic acid according to any of the above-described aspects of the invention, in conjunction with a pharmaceutically-acceptable carrier. The term “pharmaceutically-acceptable carrier” includes large, slowly metabolised macromolecules such as proteins, polysaccharides, polylactic acids, polyglycolic acids, polymeric amino acids, amino acid copolymers and inactive virus particles. Pharmaceutically acceptable salts may also be used, for example, mineral acid salts such as hydrochlorides, hydrobromides, phosphates, and sulphates, or salts of organic acids such as acetates, propionates, malonates, benzoates (see Remington's Pharmaceutical Sciences (Mack Pub. Co., N.J. 1991)). Pharmaceutically acceptable carriers in therapeutic compositions may also contain liquids such as water, saline, glycerol and ethanol. Additionally, auxiliary substances, such as wetting or emulsifying agents and pH buffering substances, may be present.

Typically, pharmaceutical compositions are prepared as injectables, either as liquid solutions or suspensions, or for application in patches. Solid forms suitable for solution in, or suspension in liquid vehicles prior to injection may also be prepared. Preparations for oral administration may also be formulated to allow for controlled release of the active compound. For administration by inhalation, the compounds of the invention may be delivered by any means known to those of the skill in the art, such as using aerosol sprays, capsules or cartridges.

According to a further aspect of the invention there is provided a method for treating a patient suffering from a disorder whose pathology is dependent upon a VEGF-mediated metabolic pathway comprising administering to a patient a therapeutically-effective amount of a protein, a nucleic acid encoding such a protein, or a pharmaceutical composition according to any one of the above-described aspects of the invention.

According to a still further aspect of the invention there is provided a genetically-modified animal, such as a transgenic animal, particularly a transgenic rodent animal, which expresses a protein according to any one of the above-described aspects of the invention. Methods for the production of genetically-modified animals are known in the art and include techniques such as modification of somatic cells, or germ line therapy. to incorporate heritable modifications (see, for example, Rajewsky et al., (1996), J Clin Invest 98, 600-3; Metzger and Feil, (1999) Curr. Opinions Biotechnology 10, 470-476; Bedell et al. (1997), Genes Dev. 11: 1143; Bedell et al (1997), Genes Dev. 11: 1-10; “Transgenic Mammals”, John Bishop (1999) Pearson Education Ltd., Harlow, Essex, for example, p228). Preferably, transgenic organisms are created using germ line gene therapy.

According to a still further embodiment of the invention, there is provided a method for inhibiting the dimerisation of a VEGF receptor, comprising bringing the receptor into contact with a protein, or functional equivalent according to any one of the embodiments of the invention described above.

Various aspects and embodiments of the present invention will now be described in more detail by way of example. It will be appreciated that modification of detail may be made without departing from the scope of the invention.

### BRIEF DESCRIPTION OF THE FIGURES

FIG. 1 gives the sequence of the Homo sapiens FLT-1 gene (acc. no. NM_{—}002019)

FIG. 2 gives the sequence of the Mus musculus FLK-1 gene (acc. no. X70842).

FIG. 3 gives the sequence of the Homo sapiens KDR/flk-1 gene (acc. no. AF035121).

FIG. 4 gives the sequence of the Mus musculus Flt-4 gene (acc. no. NM_{—}008029.

FIG. 5 shows an alignment of the most relevant parts of the fourth Ig-like domains of the FLT-1, FLK, KDR and FLT-4 protein sequences. Residues 316-447 of domain IV of FLT-1 are shown aligned to residues 312-438 of KDR, 314-440 of FLK and 315-437 of FLT-4.

FIG. 6 presents a Coomassie-stained SDS-PAGE gel showing the purification of His-Tagged Domain 4 (Construct 0) from E. coli crude soluble extract. Lane 1 shows unbound crude protein from His-tag column after incubation with 2M Urea buffer; Lane 2 shows the bound protein after elution with 400 mM imidazole; Lane 3 shows unbound crude protein from His-tag column after incubation with 4 M Urea buffer; Lane 4 shows the bound protein after elution with 400 mM imidazole; Lane 5 shows unbound crude protein from the His-tag column after incubation with 6 M Urea buffer; and Lane 6 shows the bound protein after elution with 400 mM imidazole.

FIG. 7 presents a Coomassie-stained SDS-PAGE gel showing all four constructs of Domain 4 after His-tag purification. Lanes 1 to 4 show the step-wise purification of construct 0 (14.16 kDa) with increasing concentrations of imidazole to a final elution concentration of 400 mM imidazole. Lane 1 shows unbound crude protein from construct 0; lane 2 shows eluant after 50 mM imidazole wash; lane 3 shows eluant after 100 mM imidazole wash; and lane 4 shows eluant after 40omM imidazole wash. Lanes 5 and 6 show purification of construct 1 (13.8 kDa) after a final wash step with 100 mM imidazole (lane 5) and elution with 400 mM imidazole (lane 6). Lanes 7 and 8 show purification of construct 2 (15.12 kDa) after a final wash step with 100 mM imidazole (lane 7) and elution with 400 mM imidazole (lane 8) and finally lanes 9 and 10 show purification of construct 3 (12.84 kDa) after a final wash step with 100 mM imidazole (lane 9) and elution with 400 mM imidazole (lane 10).

FIG. 8 shows the specific detection of the four Domain 4 constructs by Western blotting using an anti-sFLT-1 polyclonal antibody. Lanes 1 and 2 show construct 0, lanes 3 and 4 construct 1, lanes 5 and 6 construct 2 and lanes 7 and 8 construct 3.

FIG. 9 shows the specific detection of the four Domain 4 constructs by Western blotting using an anti-penta Histidine monoclonal antibody. Lane 1 shows construct 0, lane 2 construct 1, lane 3 construct 2 and lane 4 construct 3.

FIG. 10 presents BIACORE data of the binding of sFLT-1 in the presence and absence of all four Domain 4 constructs.

FIG. 11 presents BIACORE data of the binding of all four Domain 4 constructs on the chip.

FIG. 12 shows slot blot data of Domain 4 constructs 0, 1 and 2 screened with hybridomas 1H11, 6C5, 18G12, and 15F8.

Table 1 shows the boundaries of preferred constructs for the FLT-1, KDR, FLK and FLT-4 VEGF receptors.

Table 2 demonstrates the percentage of inhibition achieved by all four Domain 4 constructs on the binding of the sFLT-1 molecule on to the 2B2 antibody

### EXAMPLES

### Example 1

### Preparation of FLT-1 Domain 4 Molecules

Construct 0

| | |
|---|---|
| 338 | HRKQQVLETVAGKRSYRLSMKVKAFPSPEVVWLKDGLPATEKSAR |
| | YLTRGYSLIIXDVTEEDAGNYTILLSIKQSNVFKNLTATLIVNVK |
| | PQIYEKAVSSFPD 440 |

Construct 1

| | |
|---|---|
| 330 | DKAFITVKHRKQQVLETVAGKRSYRLSMKVKAFPSPEVVWLKDGL |
| | PATEKSARYLTRGYSLIIKDVTEEDAGNYTILLSIKQSNVFKNLT |
| | ATLIVNVKPQ 429 |

Construct 2

| | |
|---|---|
| 330 | DKAFITVKHRKQQVLETVAGKRSYRLSMKVKAFPSPEVVWLKDGL |
| | PATEKSARYLTRGYSLIIKDVTEEDAGNYTILLSIKQSNVFKNLT |
| | ATLIVNVKPQIYEKAVSSFPD 440 |

Construct 3

| | |
|---|---|
| 338 | HRKQQVLETVAGKRSYRLSMKVKAFPSPEVVWLKDGLPATEKSAR |
| | YLTRGYSLIIKDVTEEDAGNYTILLSIKQSNVFKNLTATLIVNV |
| | KPQ 429 |

The protein sequences of four potential Domain 4 constructs of FLT-1 are given above and contain residues 338-440 (Construct 0), residues 330-429 (Construct 1), 330-440 (Construct 2) and 338-429 (Construct 3) respectively. The underlined residues are predicted to be strands by homology alignments to'the known structures of telokin (1TLK) and domain 2 of FLT-1 (1FLT).

Any of the above constructs of the fourth Ig-like domain may be cloned by polymerase chain reaction (PCR) from the full length sFLT-1 clone using the upstream primer

Construct 0

(D4for) Sense 5′ GGGAATTCCATATGCATCGAAAACAGCAGGTGCTTGAAAC 3′, containing an NdeI site and the downstream primer

(D4rev) Antisense 5′ CGCGGATCCTTAGTCTGGAAACGATGACACGGC 3′ containing an artificial stop codon and a BamHI restriction site.

Construct 1

(Flt 39) Sense 5′CCGGTATCCATATGGATAAAGCATTCATCACTGTG3′ containing an NdeI site and the downstream primer

(Flt 40) Antisense 5′CGCGGATCCTTACTGGGGTTTCACATTGACAATTAGAG 3′ containing an artificial stop codon and a BamHI restriction site.

Construct 2

(Fit 39) Sense 5′CCGGTATCCATATGGATAAAGCATTCATCACTGTG3′ containing an NdeI site and the downstream primer

(D4rev) Antisense 5′CGCGGATCCTTAGTCTGGAAACGATGACACGGC3′ containing an artificial stop codon and BamHI restriction site.

Construct 3

(D4for) Sense 5′GGGAATTCCATATGCATCGAAAACAGCAGGTGCTTGAAAC3′ containing an NdeI site and the downstream primer

(Flt 40) Antisense 5′CGCGGATCCTTACTGGGGTTTCACATTGACAATTAGAG 3′ containing an artificial stop codon and a BamHI restriction site. The regions complementary to the FLT-1 molecule are shown in bold.

PCR products are purified with the QIAquick gel extraction kit (QIAGEN) and digested with BamHI and NdeI. The digested fragments are purified with the QIAquick PCR purification kit (Qiagen) and ligated into the E.coli vector pEE14b (Novagen) that was digested by NdeI and BamHI, followed by dephosphorylation using calf intestinal alkaline phosphatase. The presence of the insert was verified by double digestion with NdeI and BamHI.

For expression, the plasmid construct is transformed in E. coli strain BL21(DE3) carrying the inducible T7 polymerase gene. Bacterial cultures of lit of LB medium containing 200 μg/ml ampicillin are grown in shaking flasks at 37° C. up to an optical density of 0.7 at 600 nm. The culture is grown for another 4 h after addition of 50 μM isopropyl-β-D-thiogalactosidase at 25° C. Cells are harvested and the pellets frozen at −80° C.

Cells are lysed by mild sonication in 50 nM Tris-HCl, 300 mM NaCl, 10% glycerol, 0.25 mM PMSF pH 8.0. Any insoluble material is removed by centrifugation at 20000 g for 30 min at 4° C. and the histidine-tagged protein is batch absorbed onto Ni-NTA-agarose (Qiagen). The resin is washed twice with sonication buffer, followed by two washes with 50 mM Tris-HCl, 300 mM NaCl, 5 mM imidazole, 20% glycerol pH 8.0. The protein is eluted from the resin with 50 mM Tris-HCl, 100 mM NaCl, 10% glycerol, 300 mM imidazole pH 8.0.

Removal of the His-Tag is performed using 6 units of thrombin at 4° C. overnight. The untagged protein is subsequently purified from other minor contaminants on a Superdex S-100 gel-filtration column equilibrated in 20 mM Tris-HCl, 150 mM NaCl, pH 8.0. The identity of the material is confirmed by N-terminal sequencing.

Alternative Method for Expression of FLT-1 Domain 4 Molecules

For expression, the plasmid construct was transformed in E. coli strain BL21(DE3)pLysS carrying the inducible T7 RNA polymerase gene and the cells were plated onto LB agar containing 50 μg/mil ampicillin, 34 μg/ml chloramphenicol and 1% glucose and incubated overnight at 37° C. A single colony of these cells are aseptically picked from the agar plate and inoculated into 5 ml LB broth containing 50 μg/ml ampicillin, 34 μg/ml chloramphenicol and 1% glucose and grown with shaking (220 rpm) overnight at 37° C. The following morning, 1 ml of the overnight suspension is used to inoculate fresh 100 ml LB broth containing 50 μg/ml ampicillin, 34 μg/ml chloramphenicol and 1% glucose and grown with shaking at 37° C. until an optical density of 0.7 at 600 nm is reached. The cells are then pelleted by centrifugation at 3500 rpm for 15 minutes at 4° C. and resuspended in fresh 100 ml LB media containing 50 μg/ml ampicillin, 34 μg/ml chloramphenicol 1% glucose and 0.1 mM isopropyl-β-D-thiogalactosidase in order to induce protein expression. Recombinant protein is produced from the cells for 16 hours at 16° C. The cells are harvested and the pellets frozen at −80° C.

Cells lysis was performed by using Novagen's Bug Buster™ Protein Extraction Reagent following the manufacturer's instructions and both the soluble clarified protein as well as inclusion bodies are collected.

The crude soluble fraction containing the histidine-tagged Domain 4 variants was purified using an Ni-NTA agarose column (QIAGEN). Purification was performed under denaturing conditions, since the His-Tag tends to interact with the protein backbone, so making the protein inaccessible to the resin. Batch purification was performed by incubating the crude material overnight in buffer containing 6 M Urea, 10 mM Tris-HCl, 100 mM NaH₂PO₄ pH 8.0 with pre-equilibrated Ni-NTA resin at room temperature. The resin was removed by centrifugation and the unbound supernatant removed. The Ni-NTA agarose containing absorbed His-Tagged Domain 4 protein was resuspended in wash buffer containing 6M Urea, 10 mM Tris-HCl, 100 mM NaH₂PO₄, pH 8.0 and loaded onto a column fitted with a 3 μm filter. The column containing resin was step-wise washed with wash buffer containing increasing concentrations of imidazole (from 25 to 100 mM) and the fusion protein eluted from the resin with wash buffer containing 400 mM imidazole, pH 8.0. The identity of the eluted Domain 4 fusion proteins was confirmed by SDS-Coomassie staining and Western blotting using both an anti-His_Tag monoclonal antibody as well as an anti-Flt polyclonal antibody.

### Example 2

### Preliminary Analysis of Domain 4 Variants

SDS Page analysis of the Four Domain 4 Constructs

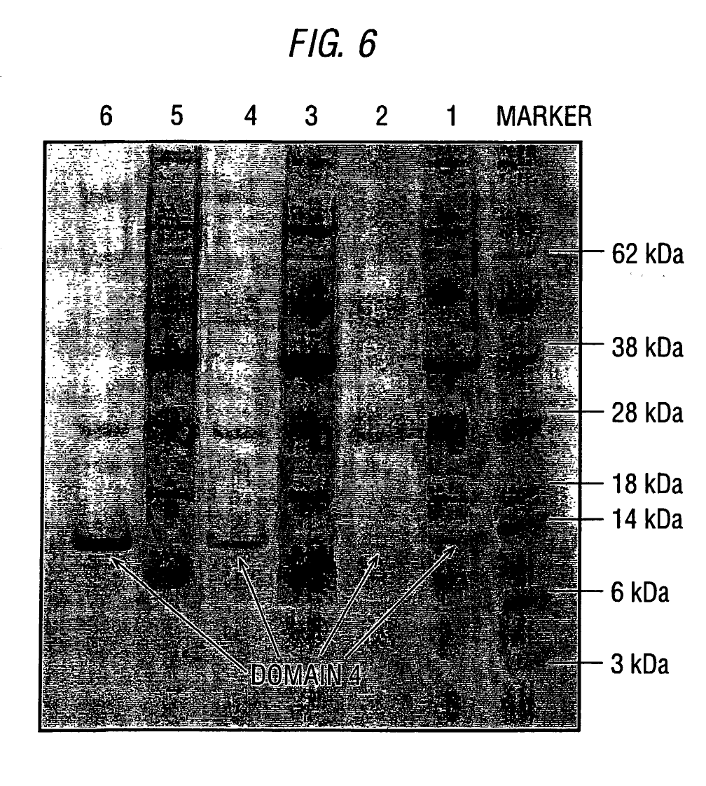

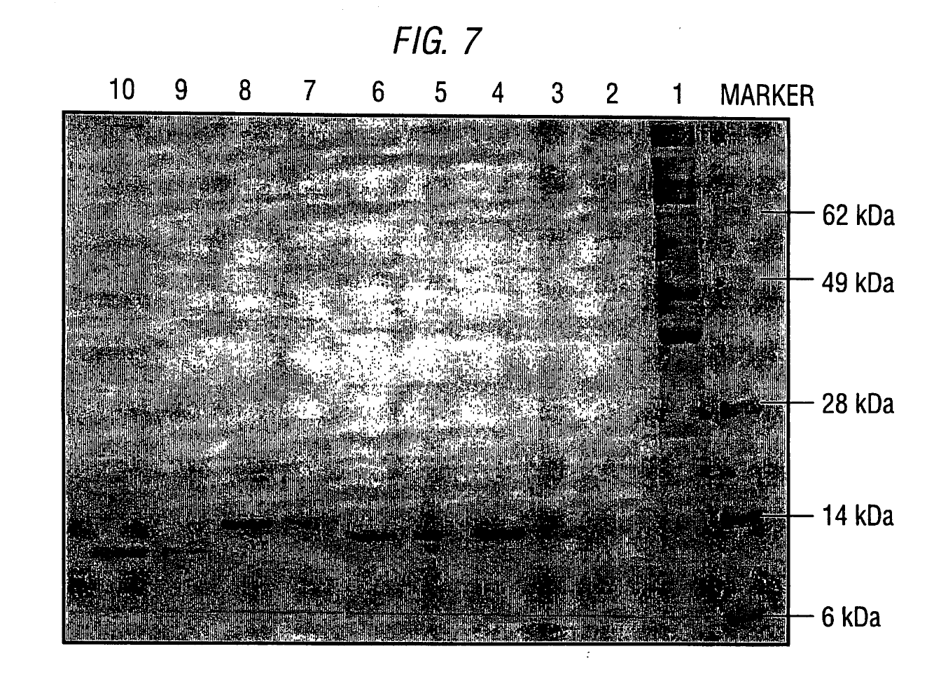

15 μl aliquots of protein samples were added to 3 μl of 4×NuPAGE LDS loading buffer (Novex) and 2 μl of 10×NuPAGE sample reducing agent (Novex), and heated at 99° C. for 5 minutes prior to loading on a 4-12% Bis-Tris SDS PAGE gel in NuPAGE MES SDS running buffer (Novex). Protein separation was carried out for 45 minutes at 200 V. Proteins were stained with Coomassie blue stain for 45 minutes and destained for 1 hour with 10% glacial acetic acid and 30% methanol. The results are shown in FIGS. 6 and 7.

Western Blotting Analysis of the Four Domain 4 Constructs

Western blotting of all four constructs was performed by using anti-sFLT-1 specific antibody as follows;

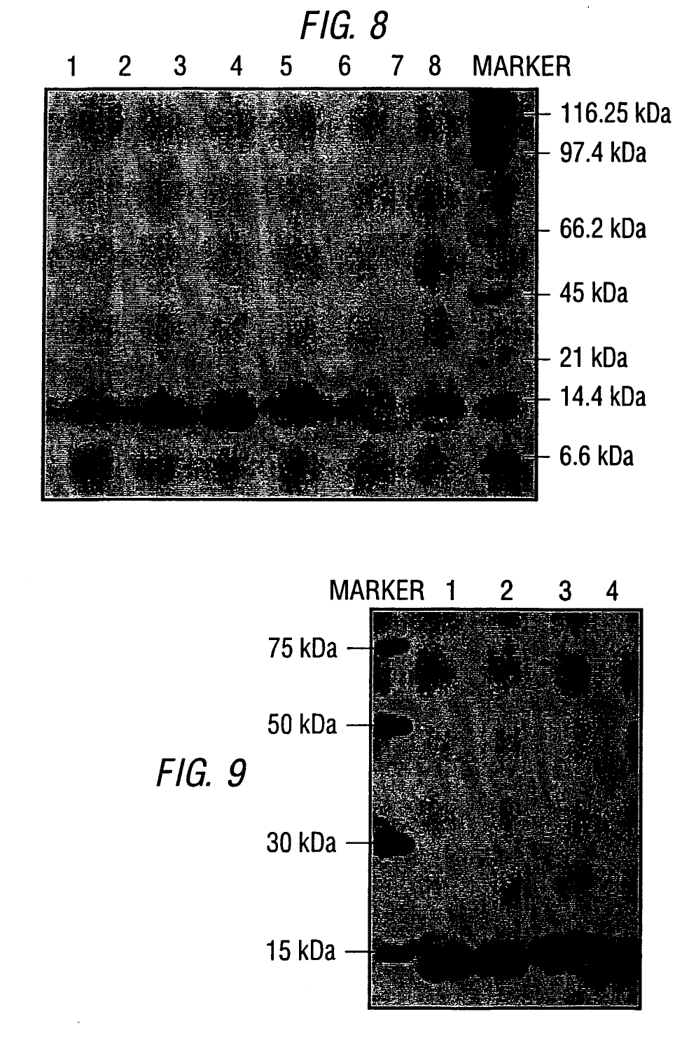

After electrophoresis, gels were transferred to polyvinylidene difluoride (PVDF) membrane in NuPAGE transfer buffer (Novex) with antioxidant and 20% methanol. The transferred membranes were blocked in phosphate buffered saline (PBS) with 1% BSA for 1 hour at room temperature prior to incubation for 1 hour in PBS with 1% bovine gamma globulin, 0.05% Tween-20 and polyclonal goat anti-sFLT-1 biotinylated antibody (0.2 ug/ml). The membranes were then washed for 5 minutes in PBS and three times in PBS-0.05% Tween for 5 minutes, and finally washed for another 5 minutes in PBS. Thereafter the membranes were incubated in PBS with 1% bovine gamma globulin, 0.05% Tween-20 and polyclonal goat anti-biotin horseradish peroxidase (HRP) labelled antibody (dilution 1/1000) for 1 hour before thoroughly washing as previously described. The membranes were then developed using Super Signal West Pico chemiluminescence substrate luminol and enhancer (Pierce) for 5 minutes prior to chemiluminescence detection reaction and exposure to X-ray film. The data are shown in FIG. 8.

Additionally, Western blots of all four constructs was performed using an anti-Penta Histidine monoclonal antibody according to the manufacturer's instructions for the antibody (QIAGEN). The results are shown in FIG. 9.

Refolding of the Four Domain 4 Fusion Protein Constructs

Refolding of the four constructs is necessary as they are purified under denaturing conditions. Refolding was performed by dialysis. The total protein concentration of the purified denatured protein was determined by BioRad protein assay and thereafter dialysed against a 50×volume of dialysis buffer containing 20 mM Tris-HCl, pH 7.4 at 4° C. for 4 hours with constant rotation. Thereafter the buffer was changed and the fusion protein dialysed overnight at 4° C. The protein concentration after refolding was measured by the BioRad protein assay according to manufacturer's instructions. Refolding was confirmed by Circular Dichroism (CD) analysis (data not shown).

All four Domain 4 variants were particularly hydrophobic and tended to fall out of solution during prolonged storage at 4° C. Out of all four constructs, constructs 1 and 2 were the least problematic, as they remained in solution even at high concentrations for few days. Construct 0 tended to precipitate very soon after refolding and concentration while construct 3 was not reliably expressed at all times.

The hydrophobic properties of the E.coli-produced Domain 4 variants may be due to lack of glycosylation. Peptide mapping experiments of sFLT-1 performed herein were consistent with one of the two potential glycosylation sites being glycosylated within Domain 4. Domain 4 contains two consensus N-glycosylation sites, 402NYT404 and 417NLT419; the peptide maps were consistent with 417NLT419 being consistently glycosylated. Interestingly, this site lies within a particularly hydrophobic region of Domain 4.

### Example 3

### Analysis of FLT-1 Domain 4 Molecules

Binding of Domain 4 on sFLT-1 is determined using Biomolecular Interaction Analysis (Biacore). (Fisher R. J. and Fivash, M. (1994) Curr. Opin. Biotech. 5, 389-395).

A monoclonal antibody (2B2) specific to Domains 1 to 3 of sFLT-1, but not Domain 4, was immobilized on the sensor chip at a concentration of 30 μg/ml in 10 mM NaAc, pH 5. 50 μg/ml of the four Domain 4 constructs in 20 mM Tris-HCl, pH 7.4 were individually pre-incubated overnight at 4° C. with 50 nM sFLT-1 in HEPES buffer (O.O1M Hepes,pH 7.4, 0.15M NaCl, 3 mM EDTA, 0.005% polysorbate 20 (v/v)). Thereafter the pre-incubated suspensions were passed across the immobilized monoclonal antibody and binding monitored by mass sensitive detection, measured in response units (RU). A control of 50 nM sFLT-1 pre-incubated overnight in HEPES buffer containing no Domain 4 was also passed across the sensor chip and its binding affinity determined. A further control was also run whereby the four Domain 4 constructs, which have not been pre-incubated with sFLT-1, were passed over the immobilized monoclonal antibody. After each run the sensor chip was regenerated in 10 mM Glycine pH 1.75 buffer ready for the next experiment.

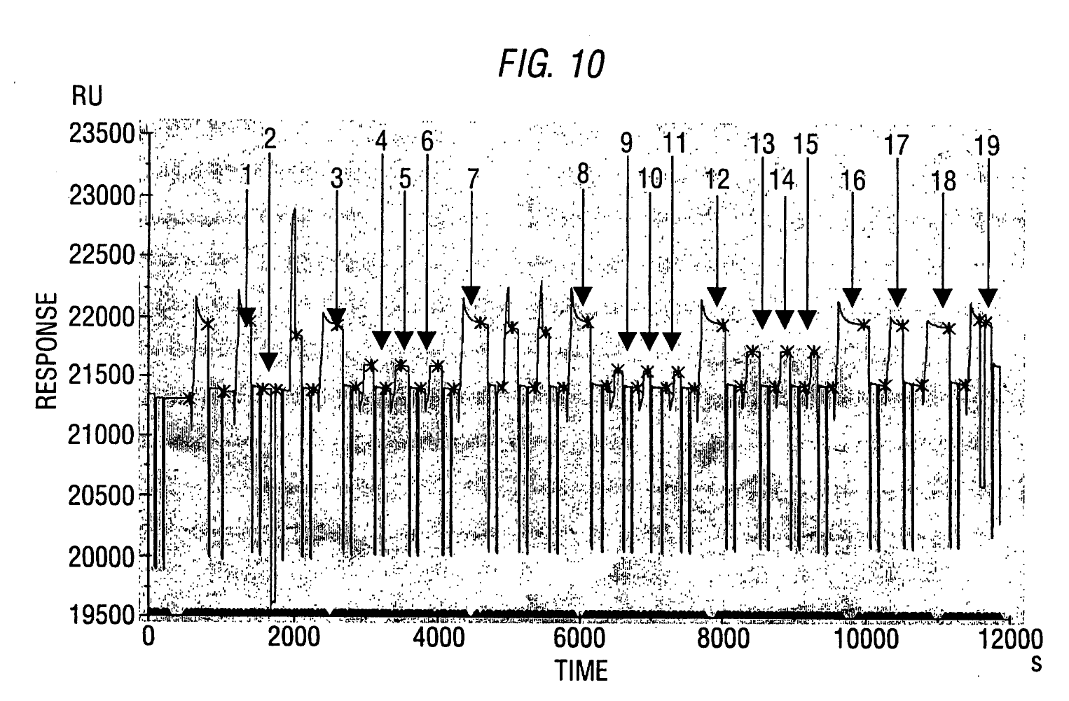

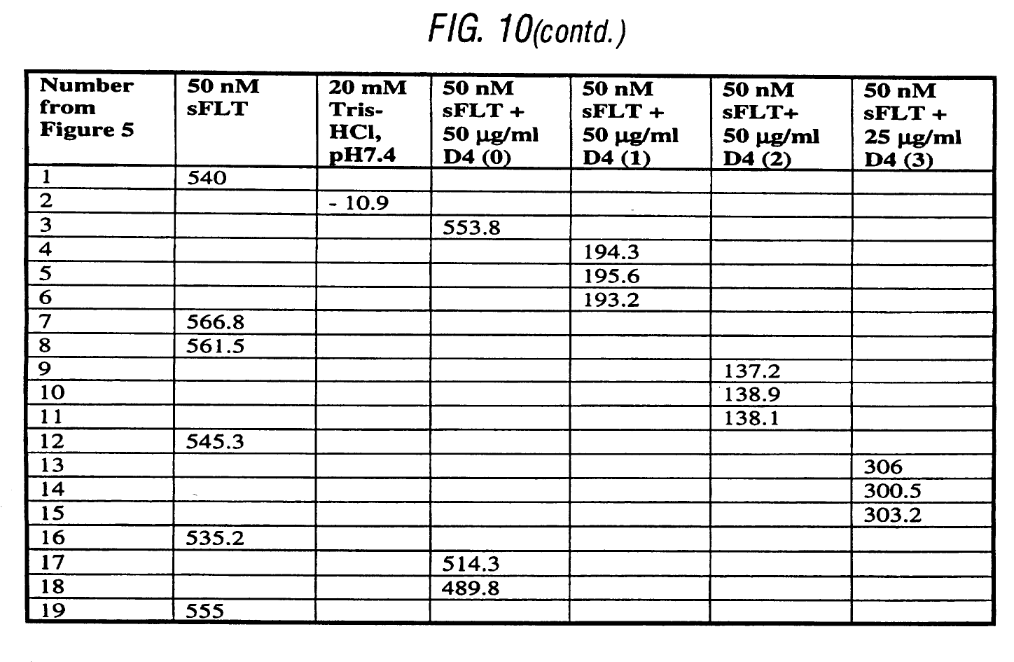

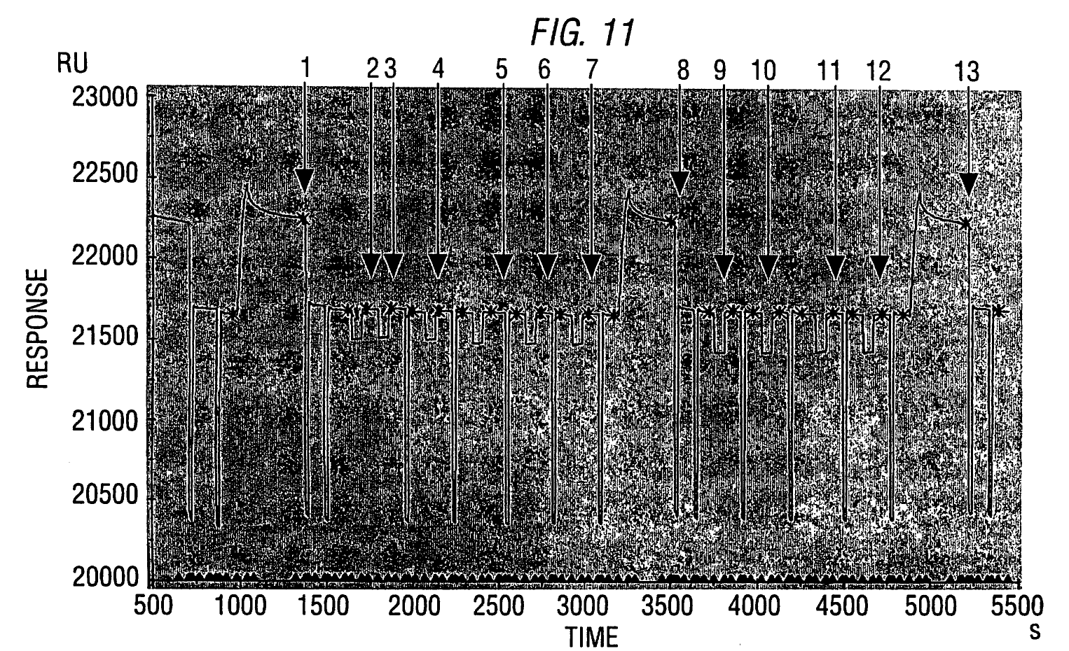

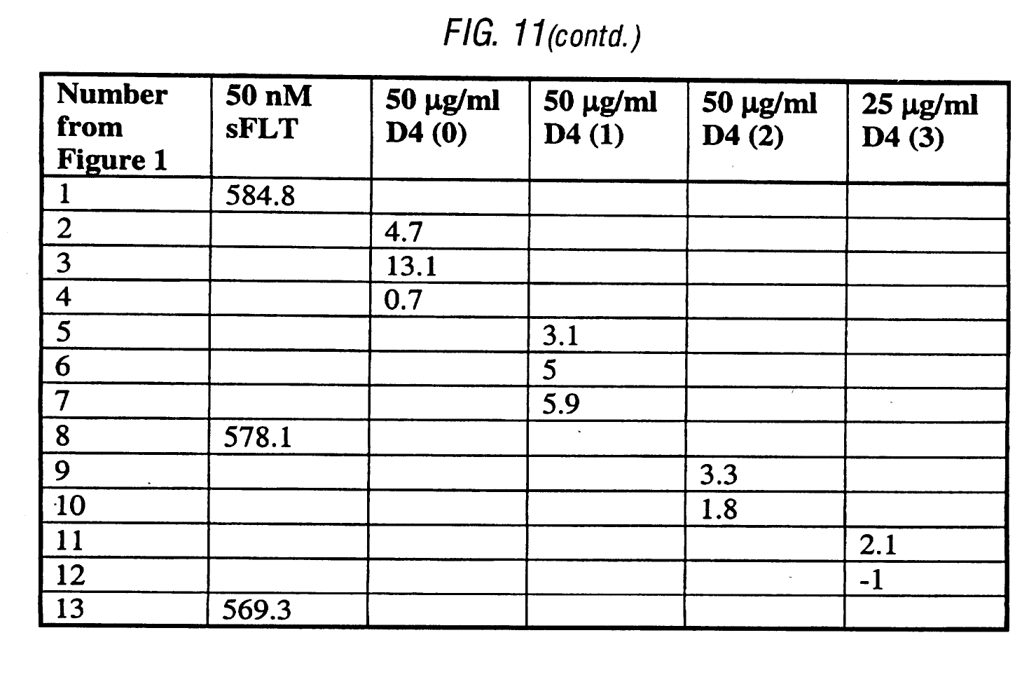

FIGS. 10 and 11 illustrate the results obtained for the four Domain 4 constructs pre-incubated with sFLT-1 prior to passing over the immobilized monoclonal antibody. In conclusion, the BIAcore data have shown that pre-incubation of sFLT-1 with either of Domain 4 constructs 1, 2 or 3 result in inhibition of sFLT-1 binding to the sensor chip (Table 1). In contrast, pre-incubation with construct 0 showed very poor inhibition (Table 1). These results clearly indicate that constructs 1, 2 and 3 interact with sFLT-1, preventing its interaction with the monoclonal antibody immobilized on the sensor chip. This interaction is a specific domain 4-sFLT-1 interaction and is not due to direct binding competition of the Domain 4 construct to the anti-body immobilised on the chip. The data in FIG. 11 have confirmed that none of the constructs bind directly onto the chip.

Alternatively, the FLT-1 molecule may be immobilised on the sensor chip and the binding affinity determined by measuring the response at the course of time at increasing concentrations of the Domain 4 protein in the presence and absence of VEGF.

The dimeric/monomeric state of Domain 4 as well as its interactions with sFLT-1 in solution may also be determined by gel filtration where molecules are fractionated by size.

High resolution gel filtration is used for separating the monomers from the dimers and heterodimers. Suitable columns are Sephacryl S-100 HR and S-200 HR (Pharmacia Biotech).

Direct binding of Domain 4 on the full-length receptor on the cell surface may be determined by receptor-binding experiments, where radioactively or fluorescent-labelled Domain 4 binds on HUVEC cells grown to confluence in 24-well tissue culture plates under the appropriate culture conditions. For competition binding studies, labelled Domain 4 may be mixed with various concentrations of unlabelled sFLT-1. After a few hours' incubation on ice, cells are washed 4 times with medium. Bound labelled Domain 4 is removed from cells by lysis with 0.1% SDS, and counts are measured in a γ counter.

The HUVEC proliferation assay (Clauss et al., 1996, J. Biol. Chem. 271 (3): 17629-17634) may be also used to determine the inhibitory effect of the Domain 4 protein molecules on receptor dimerisation and subsequent cell' proliferation. HUVECs are seeded in growth medium (EBM containing 2%FBS and GA-1000) at 5000 cells/well in 96-well cluster plates. The plates are left overnight for cell attachment/stabilisation and the cells are then treated with VEGF in the presence and absence of Domain 4 molecules at variable concentrations. Cell proliferation is measured 48 hrs after incubation by the Br-dU ELISA (Boehringer Mannheim) (Porstmann et al., (1985) J. Immunol. Methods 82: 169-179).

### Example 4

### Preparation of a Monoclonal Antibody Against Domain 4

Alternatively, an antibody, preferably a monoclonal antibody, recognising Domain 4 may be used to restrict dimerisation of the receptor. One antibody recognising Domain 4 has been produced herein and the method for its production is described below.

Three hosts were immunised with sFLT-1 produced in CHO cells in Freund's complete adjuvant, intra-muscular, followed by 3 immunisations at 2 week intervals in Freund's incomplete adjuvant subcutaneously, followed by a test bleed 1 week later each immunisation. A final intra-peritoneal immunisation was given 2 weeks later before spleen collection. 1 of the 3 hosts was chosen for fusion and 25 hybridomas were initially selected. 6 hybrids were selected for cloning and 3 clones from each hybridoma were transferred into larger plates and yielded sufficient cells for frozen storage.

The initial bleeds were screened on a 96-well plate being coated overnight with sFLT-1, 2 μg/ml in PBS, 100 μl/well at 4° C. The plate was blocked with 1% BSA in PBS pH 7.4 (200 μl/well, 60 min, 37° C.). Primary incubation was performed with 50 μl anti-serum diluted in assay buffer plus 50 μl competing antigen (MP9814 at 5 μg/ml in assay buffer) for 1 hr at 37° C. Goat anti-mouse-IgG-alkaline phosphatase labelled (1:3000 dilution) was used for detection of the primary antibody bound onto the plate. The amount of labelled-antibody bound was determined by an alkaline phosphatase assay, using 0.5 mg/ml pNPP substrate in 9.7% diethanolamine, pH 9.6.

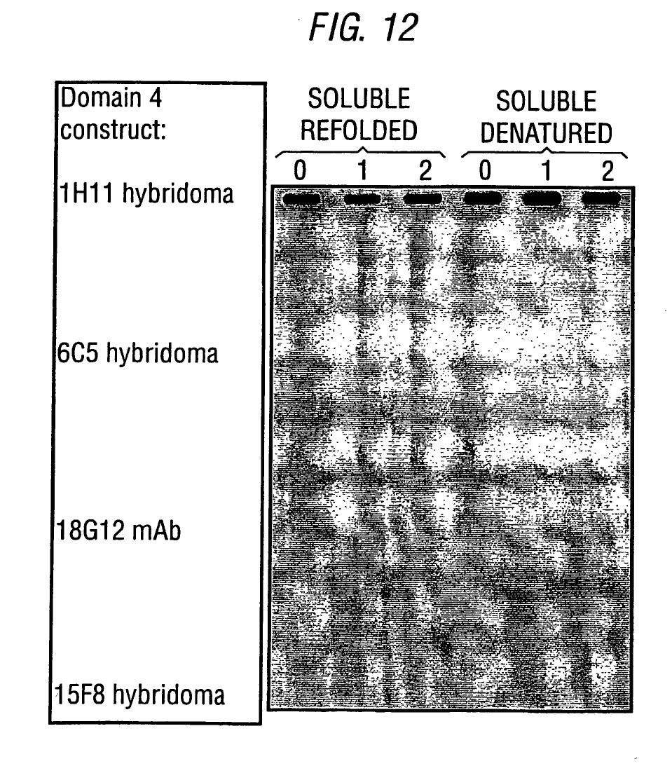

Hybridoma screening was performed as described for the screening of the test bleeds with the exception that undiluted hybridoma supernatant was used during the primary incubation in place of serum. Five hybridomas were positive for binding to sFLT-1, 5G10, 6C5, 15F8, 1H11 and 7C10 and they were screened against a number of sFLT-1 variants for binding on dot and slot blots (see FIG. 12).

Dot Blot Results

| | Hybridoma | | | | | |
|---|---|---|---|---|---|---|
| Protein | coac./neat | 5G10 | 6C5 | 15F8 | 1H11 | 7C10 |
| sFLT-1 Dom 1-5 crude | 40 μg/ml | ✓ | ✓ | low | ✓ | ✓ |
| sFLT-1 Dom 1-5 purified | 40 μg/ml | ✓ | ✓ | low | ✓ | ✓ |
| sFLT-1 Dom 1-3 crude | 40 μg/ml (?) | low | X | X | X | low |
| sFLT-1 Dom 1-3 parially purified | 40 μg/ml (?) | low | X | X | X | ? |
| sFLT-1 Dom 1-4/5 crude | neat | ✓ | ✓ | v. v. low | ✓ | ✓ |
| sFLT-1 Dom 2-3 | neat | ✓ | X | X | X | v. low |
| sFLT-1 Dom 2 fusion | neat | ✓ | X | X | X | ✓ |
| sFLT-1 Dom. 4 (construct 0) | neat | X | X | X | ✓ | ✓ |
| Blank media Excel 302 | neat | X | X | X | X | X |
| Blank media GMEM (1% serum) | neat | X | X | X | X | X |
| Wild type CHO K1 media | neat | X | X | X | X | X |
| Mock transfected media | neat | X | X | X | X | X |

All samples were loaded at volumes of 1 μl, 5 μl, 12.5 μl and 25 μl at the above concentrations, onto a nitrocellulose membrane. The membranes were blocked in a 5% skimmed milk powder solution overnight at 4° C. Primary hybridomas were prepared in 5% skimmed milk powder incubation buffer and secondary antibody was added at an 1:1000 dilution in 5% skimmed milk powder incubation buffer.

Hybridoma 1H11 is positive for Domain 4 (Construct 0). This hybridoma as well as hybridoma 6C5, 18G12 and 15F8 were tested for its, affinity to the variants of Domain 4 by slot blots. All Domain 4 constructs tested were positive for hybridoma 1H11 while hybridomas 6C5, 18G12 and 15F8 did not cross-react with any of the Domain 4 constructs and the results are shown in FIG. 12.

TABLE 1
Preferred constructs derived from the FLT-1,
FLK, KDR and FLT-4 constructs

| FLT-1 | FLK | KDR | FLT-4 |
|---|---|---|---|
| 316 to 406 | 314 to 404 | 312 to 402 | 315 to 403 |
| 316 to 407 | 314 to 405 | 312 to 403 | 315 to 404 |
| 316 to 408 | 314 to 406 | 312 to 404 | 315 to 405 |
| 316 to 409 | 314 to 407 | 312 to 405 | 315 to 406 |
| 316 to 410 | 314 to 408 | 312 to 406 | 315 to 407 |
| 316 to 411 | 314 to 409 | 312 to 407 | 315 to 408 |
| 316 to 412 | 314 to 410 | 312 to 408 | 315 to 409 |
| 316 to 413 | 314 to 411 | 312 to 409 | 315 to 410 |
| 316 to 414 | 314 to 412 | 312 to 410 | 315 to 411 |
| 316 to 415 | 314 to 413 | 312 to 411 | 315 to 412 |
| 316 to 416 | 314 to 414 | 312 to 412 | 315 to 413 |
| 316 to 417 | 314 to 415 | 312 to 413 | 315 to 414 |
| 316 to 418 | 314 to 416 | 312 to 414 | 315 to 415 |
| 316 to 419 | 314 to 417 | 312 to 415 | 315 to 416 |
| 316 to 420 | 314 to 418 | 312 to 416 | 315 to 417 |
| 316 to 421 | 314 to 419 | 312 to 417 | 315 to 418 |
| 316 to 422 | 314 to 420 | 312 to 418 | 315 to 419 |
| 316 to 423 | 314 to 421 | 312 to 419 | 315 to 420 |
| 316 to 424 | 314 to 422 | 312 to 420 | 315 to 421 |
| 316 to 425 | 314 to 423 | 312 to 421 | 315 to 422 |
| 316 to 426 | 314 to 424 | 312 to 422 | 315 to 423 |
| 316 to 427 | 314 to 425 | 312 to 423 | 315 to 424 |
| 316 to 428 | 314 to 426 | 312 to 424 | 315 to 425 |
| 316 to 429 | 314 to 427 | 312 to 425 | 315 to 426 |
| 316 to 430 | 314 to 428 | 312 to 426 | 315 to 427 |
| 316 to 431 | 314 to 429 | 312 to 427 | 315 to 428 |
| 316 to 432 | 314 to 430 | 312 to 428 | 315 to 429 |
| 316 to 433 | 314 to 431 | 312 to 429 | 315 to 430 |
| 316 to 434 | 314 to 432 | 312 to 430 | 315 to 431 |
| 316 to 435 | 314 to 433 | 312 to 431 | 315 to 432 |
| 316 to 436 | 314 to 434 | 312 to 432 | 315 to 433 |
| 316 to 437 | 314 to 435 | 312 to 433 | 315 to 434 |
| 316 to 438 | 314 to 436 | 312 to 434 | 315 to 435 |
| 316 to 439 | 314 to 437 | 312 to 435 | 315 to 436 |
| 316 to 440 | 314 to 438 | 312 to 436 | 315 to 437 |
| 316 to 441 | 314 to 439 | 312 to 437 | 316 to 403 |
| 316 to 442 | 314 to 440 | 312 to 438 | 316 to 404 |
| 316 to 443 | 315 to 404 | 313 to 402 | 316 to 405 |
| 316 to 444 | 315 to 405 | 313 to 403 | 316 to 406 |
| 316 to 445 | 315 to 406 | 313 to 404 | 316 to 407 |
| 316 to 446 | 315 to 407 | 313 to 405 | 316 to 408 |
| 316 to 447 | 315 to 408 | 313 to 406 | 316 to 409 |
| 317 to 406 | 315 to 409 | 313 to 407 | 316 to 410 |
| 317 to 407 | 315 to 410 | 313 to 408 | 316 to 411 |
| 317 to 408 | 315 to 411 | 313 to 409 | 316 to 412 |
| 317 to 409 | 315 to 412 | 313 to 410 | 316 to 413 |
| 317 to 410 | 315 to 413 | 313 to 411 | 316 to 414 |
| 317 to 411 | 315 to 414 | 313 to 412 | 316 to 415 |
| 317 to 412 | 315 to 415 | 313 to 413 | 316 to 416 |
| 317 to 413 | 315 to 416 | 313 to 414 | 316 to 417 |
| 317 to 414 | 315 to 417 | 313 to 415 | 316 to 418 |
| 317 to 415 | 315 to 418 | 313 to 416 | 316 to 419 |
| 317 to 416 | 315 to 419 | 313 to 417 | 316 to 420 |
| 317 to 417 | 315 to 420 | 313 to 418 | 316 to 421 |
| 317 to 418 | 315 to 421 | 313 to 419 | 316 to 422 |
| 317 to 419 | 315 to 422 | 313 to 420 | 316 to 423 |
| 317 to 420 | 315 to 423 | 313 to 421 | 316 to 424 |
| 317 to 421 | 315 to 424 | 313 to 422 | 316 to 425 |
| 317 to 422 | 315 to 425 | 313 to 423 | 316 to 426 |
| 317 to 423 | 315 to 426 | 313 to 424 | 316 to 427 |
| 317 to 424 | 315 to 427 | 313 to 425 | 316 to 428 |
| 317 to 425 | 315 to 428 | 313 to 426 | 316 to 429 |
| 317 to 426 | 315 to 429 | 313 to 427 | 316 to 430 |
| 317 to 427 | 315 to 430 | 313 to 428 | 316 to 431 |
| 317 to 428 | 315 to 431 | 313 to 429 | 316 to 432 |
| 317 to 429 | 315 to 432 | 313 to 430 | 316 to 433 |
| 317 to 430 | 315 to 433 | 313 to 431 | 316 to 434 |
| 317 to 431 | 315 to 434 | 313 to 432 | 316 to 435 |
| 317 to 432 | 315 to 435 | 313 to 433 | 316 to 436 |
| 317 to 433 | 315 to 436 | 313 to 434 | 316 to 437 |
| 317 to 434 | 315 to 437 | 313 to 435 | 317 to 403 |
| 317 to 435 | 315 to 438 | 313 to 436 | 317 to 404 |
| 317 to 436 | 315 to 439 | 313 to 437 | 317 to 405 |
| 317 to 437 | 315 to 440 | 313 to 438 | 317 to 406 |
| 317 to 438 | 316 to 404 | 314 to 402 | 317 to 407 |
| 317 to 439 | 316 to 405 | 314 to 403 | 317 to 408 |
| 317 to 440 | 316 to 406 | 314 to 404 | 317 to 409 |
| 317 to 441 | 316 to 407 | 314 to 405 | 317 to 410 |
| 317 to 442 | 316 to 408 | 314 to 406 | 317 to 411 |
| 317 to 443 | 316 to 409 | 314 to 407 | 317 to 412 |
| 317 to 444 | 316 to 410 | 314 to 408 | 317 to 413 |
| 317 to 445 | 316 to 411 | 314 to 409 | 317 to 414 |
| 317 to 446 | 316 to 412 | 314 to 410 | 317 to 415 |
| 317 to 447 | 316 to 413 | 314 to 411 | 317 to 416 |
| 318 to 406 | 316 to 414 | 314 to 412 | 317 to 417 |
| 318 to 407 | 316 to 415 | 314 to 413 | 317 to 418 |
| 318 to 408 | 316 to 416 | 314 to 414 | 317 to 419 |
| 318 to 409 | 316 to 417 | 314 to 415 | 317 to 420 |
| 318 to 410 | 316 to 418 | 314 to 416 | 317 to 421 |
| 318 to 411 | 316 to 419 | 314 to 417 | 317 to 422 |
| 318 to 412 | 316 to 420 | 314 to 418 | 317 to 423 |
| 318 to 413 | 316 to 421 | 314 to 419 | 317 to 424 |
| 318 to 414 | 316 to 422 | 314 to 420 | 317 to 425 |
| 318 to 415 | 316 to 423 | 314 to 421 | 317 to 426 |
| 318 to 416 | 316 to 424 | 314 to 422 | 317 to 427 |
| 318 to 417 | 316 to 425 | 314 to 423 | 317 to 428 |
| 318 to 418 | 316 to 426 | 314 to 424 | 317 to 429 |
| 318 to 419 | 316 to 427 | 314 to 425 | 317 to 430 |
| 318 to 420 | 316 to 428 | 314 to 426 | 317 to 431 |
| 318 to 421 | 316 to 429 | 314 to 427 | 317 to 432 |
| 318 to 422 | 316 to 430 | 314 to 428 | 317 to 433 |
| 318 to 423 | 316 to 431 | 314 to 429 | 317 to 434 |
| 318 to 424 | 316 to 432 | 314 to 430 | 317 to 435 |
| 318 to 425 | 316 to 433 | 314 to 431 | 317 to 436 |
| 318 to 426 | 316 to 434 | 314 to 432 | 317 to 437 |
| 318 to 427 | 316 to 435 | 314 to 433 | 318 to 403 |
| 318 to 428 | 316 to 436 | 314 to 434 | 318 to 404 |
| 318 to 429 | 316 to 437 | 314 to 435 | 318 to 405 |
| 318 to 430 | 316 to 438 | 314 to 436 | 318 to 406 |
| 318 to 431 | 316 to 439 | 314 to 437 | 318 to 407 |
| 318 to 432 | 316 to 440 | 314 to 438 | 318 to 408 |
| 318 to 433 | 317 to 404 | 315 to 402 | 318 to 409 |
| 318 to 434 | 317 to 405 | 315 to 403 | 318 to 410 |
| 318 to 435 | 317 to 406 | 315 to 404 | 318 to 411 |
| 318 to 436 | 317 to 407 | 315 to 405 | 318 to 412 |
| 318 to 437 | 317 to 408 | 315 to 406 | 318 to 413 |
| 318 to 438 | 317 to 409 | 315 to 407 | 318 to 414 |
| 318 to 439 | 317 to 410 | 315 to 408 | 318 to 415 |
| 318 to 440 | 317 to 411 | 315 to 409 | 318 to 416 |
| 318 to 441 | 317 to 412 | 315 to 410 | 318 to 417 |
| 318 to 442 | 317 to 413 | 315 to 411 | 318 to 418 |
| 318 to 443 | 317 to 414 | 315 to 412 | 318 to 419 |
| 318 to 444 | 317 to 415 | 315 to 413 | 318 to 420 |
| 318 to 445 | 317 to 416 | 315 to 414 | 318 to 421 |
| 318 to 446 | 317 to 417 | 315 to 415 | 318 to 422 |
| 318 to 447 | 317 to 418 | 315 to 416 | 318 to 423 |
| 319 to 406 | 317 to 419 | 315 to 417 | 318 to 424 |
| 319 to 407 | 317 to 420 | 315 to 418 | 318 to 425 |
| 319 to 408 | 317 to 421 | 315 to 419 | 318 to 426 |
| 319 to 409 | 317 to 422 | 315 to 420 | 318 to 427 |
| 319 to 410 | 317 to 423 | 315 to 421 | 318 to 428 |
| 319 to 411 | 317 to 424 | 315 to 422 | 318 to 429 |
| 319 to 412 | 317 to 425 | 315 to 423 | 318 to 430 |
| 319 to 413 | 317 to 426 | 315 to 424 | 318 to 431 |
| 319 to 414 | 317 to 427 | 315 to 425 | 318 to 432 |
| 319 to 415 | 317 to 428 | 315 to 426 | 318 to 433 |
| 319 to 416 | 317 to 429 | 315 to 427 | 318 to 434 |
| 319 to 417 | 317 to 430 | 315 to 428 | 318 to 435 |
| 319 to 418 | 317 to 431 | 315 to 429 | 318 to 436 |
| 319 to 419 | 317 to 432 | 315 to 430 | 318 to 437 |
| 319 to 420 | 317 to 433 | 315 to 431 | 319 to 403 |
| 319 to 421 | 317 to 434 | 315 to 432 | 319 to 404 |
| 319 to 422 | 317 to 435 | 315 to 433 | 319 to 405 |
| 319 to 423 | 317 to 436 | 315 to 434 | 319 to 406 |
| 319 to 424 | 317 to 437 | 315 to 435 | 319 to 407 |
| 319 to 425 | 317 to 438 | 315 to 436 | 319 to 408 |
| 319 to 426 | 317 to 439 | 315 to 437 | 319 to 409 |
| 319 to 427 | 317 to 440 | 315 to 438 | 319 to 410 |
| 319 to 428 | 318 to 404 | 316 to 402 | 319 to 411 |
| 319 to 429 | 318 to 405 | 316 to 403 | 319 to 412 |
| 319 to 430 | 318 to 406 | 316 to 404 | 319 to 413 |
| 319 to 431 | 318 to 407 | 316 to 405 | 319 to 414 |
| 319 to 432 | 318 to 408 | 316 to 406 | 319 to 415 |
| 319 to 433 | 318 to 409 | 316 to 407 | 319 to 416 |
| 319 to 434 | 318 to 410 | 316 to 408 | 319 to 417 |
| 319 to 435 | 318 to 411 | 316 to 409 | 319 to 418 |
| 319 to 436 | 318 to 412 | 316 to 410 | 319 to 419 |
| 319 to 437 | 318 to 413 | 316 to 411 | 319 to 420 |
| 319 to 438 | 318 to 414 | 316 to 412 | 319 to 421 |
| 319 to 439 | 318 to 415 | 316 to 413 | 319 to 422 |
| 319 to 440 | 318 to 416 | 316 to 414 | 319 to 423 |
| 319 to 441 | 318 to 417 | 316 to 415 | 319 to 424 |
| 319 to 442 | 318 to 418 | 316 to 416 | 319 to 425 |
| 319 to 443 | 318 to 419 | 316 to 417 | 319 to 426 |
| 319 to 444 | 318 to 420 | 316 to 418 | 319 to 427 |
| 319 to 445 | 318 to 421 | 316 to 419 | 319 to 428 |
| 319 to 446 | 318 to 422 | 316 to 420 | 319 to 429 |
| 319 to 447 | 318 to 423 | 316 to 421 | 319 to 430 |
| 320 to 406 | 318 to 424 | 316 to 422 | 319 to 431 |
| 320 to 407 | 318 to 425 | 316 to 423 | 319 to 432 |
| 320 to 408 | 318 to 426 | 316 to 424 | 319 to 433 |
| 320 to 409 | 318 to 427 | 316 to 425 | 319 to 434 |
| 320 to 410 | 318 to 428 | 316 to 426 | 319 to 435 |
| 320 to 411 | 318 to 429 | 316 to 427 | 319 to 436 |
| 320 to 412 | 318 to 430 | 316 to 428 | 319 to 437 |
| 320 to 413 | 318 to 431 | 316 to 429 | 320 to 403 |
| 320 to 414 | 318 to 432 | 316 to 430 | 320 to 404 |
| 320 to 415 | 318 to 433 | 316 to 431 | 320 to 405 |
| 320 to 416 | 318 to 434 | 316 to 432 | 320 to 406 |
| 320 to 417 | 318 to 435 | 316 to 433 | 320 to 407 |
| 320 to 418 | 318 to 436 | 316 to 434 | 320 to 408 |
| 320 to 419 | 318 to 437 | 316 to 435 | 320 to 409 |
| 320 to 420 | 318 to 438 | 316 to 436 | 320 to 410 |
| 320 to 421 | 318 to 439 | 316 to 437 | 320 to 411 |
| 320 to 422 | 318 to 440 | 316 to 438 | 320 to 412 |
| 320 to 423 | 319 to 404 | 317 to 402 | 320 to 413 |
| 320 to 424 | 319 to 405 | 317 to 403 | 320 to 414 |
| 320 to 425 | 319 to 406 | 317 to 404 | 320 to 415 |
| 320 to 426 | 319 to 407 | 317 to 405 | 320 to 416 |
| 320 to 427 | 319 to 408 | 317 to 406 | 320 to 417 |
| 320 to 428 | 319 to 409 | 317 to 407 | 320 to 418 |
| 320 to 429 | 319 to 410 | 317 to 408 | 320 to 419 |
| 320 to 430 | 319 to 411 | 317 to 409 | 320 to 420 |
| 320 to 431 | 319 to 412 | 317 to 410 | 320 to 421 |
| 320 to 432 | 319 to 413 | 317 to 411 | 320 to 422 |
| 320 to 433 | 319 to 414 | 317 to 412 | 320 to 423 |
| 320 to 434 | 319 to 415 | 317 to 413 | 320 to 424 |
| 320 to 435 | 319 to 416 | 317 to 414 | 320 to 425 |
| 320 to 436 | 319 to 417 | 317 to 415 | 320 to 426 |
| 320 to 437 | 319 to 418 | 317 to 416 | 320 to 427 |
| 320 to 438 | 319 to 419 | 317 to 417 | 320 to 428 |
| 320 to 439 | 319 to 420 | 317 to 418 | 320 to 429 |
| 320 to 440 | 319 to 421 | 317 to 419 | 320 to 430 |
| 320 to 441 | 319 to 422 | 317 to 420 | 320 to 431 |
| 320 to 442 | 319 to 423 | 317 to 421 | 320 to 432 |
| 320 to 443 | 319 to 424 | 317 to 422 | 320 to 433 |
| 320 to 444 | 319 to 425 | 317 to 423 | 320 to 434 |
| 320 to 445 | 319 to 426 | 317 to 424 | 320 to 435 |
| 320 to 446 | 319 to 427 | 317 to 425 | 320 to 436 |
| 320 to 447 | 319 to 428 | 317 to 426 | 320 to 437 |
| 321 to 406 | 319 to 429 | 317 to 427 | 321 to 403 |
| 321 to 407 | 319 to 430 | 317 to 428 | 321 to 404 |
| 321 to 408 | 319 to 431 | 317 to 429 | 321 to 405 |
| 321 to 409 | 319 to 432 | 317 to 430 | 321 to 406 |
| 321 to 410 | 319 to 433 | 317 to 431 | 321 to 407 |
| 321 to 411 | 319 to 434 | 317 to 432 | 321 to 408 |
| 321 to 412 | 319 to 435 | 317 to 433 | 321 to 409 |
| 321 to 413 | 319 to 436 | 317 to 434 | 321 to 410 |
| 321 to 414 | 319 to 437 | 317 to 435 | 321 to 411 |
| 321 to 415 | 319 to 438 | 317 to 436 | 321 to 412 |
| 321 to 416 | 319 to 439 | 317 to 437 | 321 to 413 |
| 321 to 417 | 319 to 440 | 317 to 438 | 321 to 414 |
| 321 to 418 | 320 to 404 | 318 to 402 | 321 to 415 |
| 321 to 419 | 320 to 405 | 318 to 403 | 321 to 416 |
| 321 to 420 | 320 to 406 | 318 to 404 | 321 to 417 |
| 321 to 421 | 320 to 407 | 318 to 405 | 321 to 418 |
| 321 to 422 | 320 to 408 | 318 to 406 | 321 to 419 |
| 321 to 423 | 320 to 409 | 318 to 407 | 321 to 420 |
| 321 to 424 | 320 to 410 | 318 to 408 | 321 to 421 |
| 321 to 425 | 320 to 411 | 318 to 409 | 321 to 422 |
| 321 to 426 | 320 to 412 | 318 to 410 | 321 to 423 |
| 321 to 427 | 320 to 413 | 318 to 411 | 321 to 424 |
| 321 to 428 | 320 to 414 | 318 to 412 | 321 to 425 |
| 321 to 429 | 320 to 415 | 318 to 413 | 321 to 426 |
| 321 to 430 | 320 to 416 | 318 to 414 | 321 to 427 |
| 321 to 431 | 320 to 417 | 318 to 415 | 321 to 428 |
| 321 to 432 | 320 to 418 | 318 to 416 | 321 to 429 |
| 321 to 433 | 320 to 419 | 318 to 417 | 321 to 430 |
| 321 to 434 | 320 to 420 | 318 to 418 | 321 to 431 |
| 321 to 435 | 320 to 421 | 318 to 419 | 321 to 432 |
| 321 to 436 | 320 to 422 | 318 to 420 | 321 to 433 |
| 321 to 437 | 320 to 423 | 318 to 421 | 321 to 434 |
| 321 to 438 | 320 to 424 | 318 to 422 | 321 to 435 |
| 321 to 439 | 320 to 425 | 318 to 423 | 321 to 436 |
| 321 to 440 | 320 to 426 | 318 to 424 | 321 to 437 |
| 321 to 441 | 320 to 427 | 318 to 425 | 322 to 403 |
| 321 to 442 | 320 to 428 | 318 to 426 | 322 to 404 |
| 321 to 443 | 320 to 429 | 318 to 427 | 322 to 405 |
| 321 to 444 | 320 to 430 | 318 to 428 | 322 to 406 |
| 321 to 445 | 320 to 431 | 318 to 429 | 322 to 407 |
| 321 to 446 | 320 to 432 | 318 to 430 | 322 to 408 |
| 321 to 447 | 320 to 433 | 318 to 431 | 322 to 409 |
| 322 to 406 | 320 to 434 | 318 to 432 | 322 to 410 |
| 322 to 407 | 320 to 435 | 318 to 433 | 322 to 411 |
| 322 to 408 | 320 to 436 | 318 to 434 | 322 to 412 |
| 322 to 409 | 320 to 437 | 318 to 435 | 322 to 413 |
| 322 to 410 | 320 to 438 | 318 to 436 | 322 to 414 |
| 322 to 411 | 320 to 439 | 318 to 437 | 322 to 415 |
| 322 to 412 | 320 to 440 | 318 to 438 | 322 to 416 |
| 322 to 413 | 321 to 404 | 319 to 402 | 322 to 417 |
| 322 to 414 | 321 to 405 | 319 to 403 | 322 to 418 |
| 322 to 415 | 321 to 406 | 319 to 404 | 322 to 419 |
| 322 to 416 | 321 to 407 | 319 to 405 | 322 to 420 |
| 322 to 417 | 321 to 408 | 319 to 406 | 322 to 421 |
| 322 to 418 | 321 to 409 | 319 to 407 | 322 to 422 |
| 322 to 419 | 321 to 410 | 319 to 408 | 322 to 423 |
| 322 to 420 | 321 to 411 | 319 to 409 | 322 to 424 |
| 322 to 421 | 321 to 412 | 319 to 410 | 322 to 425 |
| 322 to 422 | 321 to 413 | 319 to 411 | 322 to 426 |
| 322 to 423 | 321 to 414 | 319 to 412 | 322 to 427 |
| 322 to 424 | 321 to 415 | 319 to 413 | 322 to 428 |
| 322 to 425 | 321 to 416 | 319 to 414 | 322 to 429 |
| 322 to 426 | 321 to 417 | 319 to 415 | 322 to 430 |
| 322 to 427 | 321 to 418 | 319 to 416 | 322 to 431 |
| 322 to 428 | 321 to 419 | 319 to 417 | 322 to 432 |
| 322 to 429 | 321 to 420 | 319 to 418 | 322 to 433 |
| 322 to 430 | 321 to 421 | 319 to 419 | 322 to 434 |
| 322 to 431 | 321 to 422 | 319 to 420 | 322 to 435 |
| 322 to 432 | 321 to 423 | 319 to 421 | 322 to 436 |
| 322 to 433 | 321 to 424 | 319 to 422 | 322 to 437 |
| 322 to 434 | 321 to 425 | 319 to 423 | 323 to 403 |
| 322 to 435 | 321 to 426 | 319 to 424 | 323 to 404 |
| 322 to 436 | 321 to 427 | 319 to 425 | 323 to 405 |
| 322 to 437 | 321 to 428 | 319 to 426 | 323 to 406 |
| 322 to 438 | 321 to 429 | 319 to 427 | 323 to 407 |
| 322 to 439 | 321 to 430 | 319 to 428 | 323 to 408 |
| 322 to 440 | 321 to 431 | 319 to 429 | 323 to 409 |
| 322 to 441 | 321 to 432 | 319 to 430 | 323 to 410 |
| 322 to 442 | 321 to 433 | 319 to 431 | 323 to 411 |
| 322 to 443 | 321 to 434 | 319 to 432 | 323 to 412 |
| 322 to 444 | 321 to 435 | 319 to 433 | 323 to 413 |
| 322 to 445 | 321 to 436 | 319 to 434 | 323 to 414 |
| 322 to 446 | 321 to 437 | 319 to 435 | 323 to 415 |
| 322 to 447 | 321 to 438 | 319 to 436 | 323 to 416 |
| 323 to 406 | 321 to 439 | 319 to 437 | 323 to 417 |
| 323 to 407 | 321 to 440 | 319 to 438 | 323 to 418 |
| 323 to 408 | 322 to 404 | 320 to 402 | 323 to 419 |
| 323 to 409 | 322 to 405 | 320 to 403 | 323 to 420 |
| 323 to 410 | 322 to 406 | 320 to 404 | 323 to 421 |
| 323 to 411 | 322 to 407 | 320 to 405 | 323 to 422 |
| 323 to 412 | 322 to 408 | 320 to 406 | 323 to 423 |
| 323 to 413 | 322 to 409 | 320 to 407 | 323 to 424 |
| 323 to 414 | 322 to 410 | 320 to 408 | 323 to 425 |
| 323 to 415 | 322 to 411 | 320 to 409 | 323 to 426 |
| 323 to 416 | 322 to 412 | 320 to 410 | 323 to 427 |
| 323 to 417 | 322 to 413 | 320 to 411 | 323 to 428 |
| 323 to 418 | 322 to 414 | 320 to 412 | 323 to 429 |
| 323 to 419 | 322 to 415 | 320 to 413 | 323 to 430 |
| 323 to 420 | 322 to 416 | 320 to 414 | 323 to 431 |
| 323 to 421 | 322 to 417 | 320 to 415 | 323 to 432 |
| 323 to 422 | 322 to 418 | 320 to 416 | 323 to 433 |
| 323 to 423 | 322 to 419 | 320 to 417 | 323 to 434 |
| 323 to 424 | 322 to 420 | 320 to 418 | 323 to 435 |
| 323 to 425 | 322 to 421 | 320 to 419 | 323 to 436 |
| 323 to 426 | 322 to 422 | 320 to 420 | 323 to 437 |
| 323 to 427 | 322 to 423 | 320 to 421 | 324 to 403 |
| 323 to 428 | 322 to 424 | 320 to 422 | 324 to 404 |
| 323 to 429 | 322 to 425 | 320 to 423 | 324 to 405 |
| 323 to 430 | 322 to 426 | 320 to 424 | 324 to 406 |
| 323 to 431 | 322 to 427 | 320 to 425 | 324 to 407 |
| 323 to 432 | 322 to 428 | 320 to 426 | 324 to 408 |
| 323 to 433 | 322 to 429 | 320 to 427 | 324 to 409 |
| 323 to 434 | 322 to 430 | 320 to 428 | 324 to 410 |
| 323 to 435 | 322 to 431 | 320 to 429 | 324 to 411 |
| 323 to 436 | 322 to 432 | 320 to 430 | 324 to 412 |
| 323 to 437 | 322 to 433 | 320 to 431 | 324 to 413 |
| 323 to 438 | 322 to 434 | 320 to 432 | 324 to 414 |
| 323 to 439 | 322 to 435 | 320 to 433 | 324 to 415 |
| 323 to 440 | 322 to 436 | 320 to 434 | 324 to 416 |
| 323 to 441 | 322 to 437 | 320 to 435 | 324 to 417 |
| 323 to 442 | 322 to 438 | 320 to 436 | 324 to 418 |
| 323 to 443 | 322 to 439 | 320 to 437 | 324 to 419 |
| 323 to 444 | 322 to 440 | 320 to 438 | 324 to 420 |
| 323 to 445 | 323 to 404 | 321 to 402 | 324 to 421 |
| 323 to 446 | 323 to 405 | 321 to 403 | 324 to 422 |
| 323 to 447 | 323 to 406 | 321 to 404 | 324 to 423 |
| 324 to 406 | 323 to 407 | 321 to 405 | 324 to 424 |
| 324 to 407 | 323 to 408 | 321 to 406 | 324 to 425 |
| 324 to 408 | 323 to 409 | 321 to 407 | 324 to 426 |
| 324 to 409 | 323 to 410 | 321 to 408 | 324 to 427 |
| 324 to 410 | 323 to 411 | 321 to 409 | 324 to 428 |
| 324 to 411 | 323 to 412 | 321 to 410 | 324 to 429 |
| 324 to 412 | 323 to 413 | 321 to 411 | 324 to 430 |
| 324 to 413 | 323 to 414 | 321 to 412 | 324 to 431 |
| 324 to 414 | 323 to 415 | 321 to 413 | 324 to 432 |
| 324 to 415 | 323 to 416 | 321 to 414 | 324 to 433 |
| 324 to 416 | 323 to 417 | 321 to 415 | 324 to 434 |
| 324 to 417 | 323 to 418 | 321 to 416 | 324 to 435 |
| 324 to 418 | 323 to 419 | 321 to 417 | 324 to 436 |
| 324 to 419 | 323 to 420 | 321 to 418 | 324 to 437 |
| 324 to 420 | 323 to 421 | 321 to 419 | 325 to 403 |
| 324 to 421 | 323 to 422 | 321 to 420 | 325 to 404 |
| 324 to 422 | 323 to 423 | 321 to 421 | 325 to 405 |
| 324 to 423 | 323 to 424 | 321 to 422 | 325 to 406 |
| 324 to 424 | 323 to 425 | 321 to 423 | 325 to 407 |
| 324 to 425 | 323 to 426 | 321 to 424 | 325 to 408 |
| 324 to 426 | 323 to 427 | 321 to 425 | 325 to 409 |
| 324 to 427 | 323 to 428 | 321 to 426 | 325 to 410 |
| 324 to 428 | 323 to 429 | 321 to 427 | 325 to 411 |
| 324 to 429 | 323 to 430 | 321 to 428 | 325 to 412 |
| 324 to 430 | 323 to 431 | 321 to 429 | 325 to 413 |
| 324 to 431 | 323 to 432 | 321 to 430 | 325 to 414 |
| 324 to 432 | 323 to 433 | 321 to 431 | 325 to 415 |
| 324 to 433 | 323 to 434 | 321 to 432 | 325 to 416 |
| 324 to 434 | 323 to 435 | 321 to 433 | 325 to 417 |
| 324 to 435 | 323 to 436 | 321 to 434 | 325 to 418 |
| 324 to 436 | 323 to 437 | 321 to 435 | 325 to 419 |
| 324 to 437 | 323 to 438 | 321 to 436 | 325 to 420 |
| 324 to 438 | 323 to 439 | 321 to 437 | 325 to 421 |
| 324 to 439 | 323 to 440 | 321 to 438 | 325 to 422 |
| 324 to 440 | 324 to 404 | 322 to 402 | 325 to 423 |
| 324 to 441 | 324 to 405 | 322 to 403 | 325 to 424 |
| 324 to 442 | 324 to 406 | 322 to 404 | 325 to 425 |
| 324 to 443 | 324 to 407 | 322 to 405 | 325 to 426 |
| 324 to 444 | 324 to 408 | 322 to 406 | 325 to 427 |
| 324 to 445 | 324 to 409 | 322 to 407 | 325 to 428 |
| 324 to 446 | 324 to 410 | 322 to 408 | 325 to 429 |
| 324 to 447 | 324 to 411 | 322 to 409 | 325 to 430 |
| 325 to 406 | 324 to 412 | 322 to 410 | 325 to 431 |
| 325 to 407 | 324 to 413 | 322 to 411 | 325 to 432 |
| 325 to 408 | 324 to 414 | 322 to 412 | 325 to 433 |
| 325 to 409 | 324 to 415 | 322 to 413 | 325 to 434 |
| 325 to 410 | 324 to 416 | 322 to 414 | 325 to 435 |
| 325 to 411 | 324 to 417 | 322 to 415 | 325 to 436 |
| 325 to 412 | 324 to 418 | 322 to 416 | 325 to 437 |
| 325 to 413 | 324 to 419 | 322 to 417 | 326 to 403 |
| 325 to 414 | 324 to 420 | 322 to 418 | 326 to 404 |
| 325 to 415 | 324 to 421 | 322 to 419 | 326 to 405 |
| 325 to 416 | 324 to 422 | 322 to 420 | 326 to 406 |
| 325 to 417 | 324 to 423 | 322 to 421 | 326 to 407 |
| 325 to 418 | 324 to 424 | 322 to 422 | 326 to 408 |
| 325 to 419 | 324 to 425 | 322 to 423 | 326 to 409 |
| 325 to 420 | 324 to 426 | 322 to 424 | 326 to 410 |
| 325 to 421 | 324 to 427 | 322 to 425 | 326 to 411 |
| 325 to 422 | 324 to 428 | 322 to 426 | 326 to 412 |
| 325 to 423 | 324 to 429 | 322 to 427 | 326 to 413 |
| 325 to 424 | 324 to 430 | 322 to 428 | 326 to 414 |
| 325 to 425 | 324 to 431 | 322 to 429 | 326 to 415 |
| 325 to 426 | 324 to 432 | 322 to 430 | 326 to 416 |
| 325 to 427 | 324 to 433 | 322 to 431 | 326 to 417 |
| 325 to 428 | 324 to 434 | 322 to 432 | 326 to 418 |
| 325 to 429 | 324 to 435 | 322 to 433 | 326 to 419 |
| 325 to 430 | 324 to 436 | 322 to 434 | 326 to 420 |
| 325 to 431 | 324 to 437 | 322 to 435 | 326 to 421 |
| 325 to 432 | 324 to 438 | 322 to 436 | 326 to 422 |
| 325 to 433 | 324 to 439 | 322 to 437 | 326 to 423 |
| 325 to 434 | 324 to 440 | 322 to 438 | 326 to 424 |
| 325 to 435 | 325 to 404 | 323 to 402 | 326 to 425 |
| 325 to 436 | 325 to 405 | 323 to 403 | 326 to 426 |
| 325 to 437 | 325 to 406 | 323 to 404 | 326 to 427 |
| 325 to 438 | 325 to 407 | 323 to 405 | 326 to 428 |
| 325 to 439 | 325 to 408 | 323 to 406 | 326 to 429 |
| 325 to 440 | 325 to 409 | 323 to 407 | 326 to 430 |
| 325 to 441 | 325 to 410 | 323 to 408 | 326 to 431 |
| 325 to 442 | 325 to 411 | 323 to 409 | 326 to 432 |
| 325 to 443 | 325 to 412 | 323 to 410 | 326 to 433 |
| 325 to 444 | 325 to 413 | 323 to 411 | 326 to 434 |
| 325 to 445 | 325 to 414 | 323 to 412 | 326 to 435 |
| 325 to 446 | 325 to 415 | 323 to 413 | 326 to 436 |
| 325 to 447 | 325 to 416 | 323 to 414 | 326 to 437 |
| 326 to 406 | 325 to 417 | 323 to 415 | 327 to 403 |
| 326 to 407 | 325 to 418 | 323 to 416 | 327 to 404 |
| 326 to 408 | 325 to 419 | 323 to 417 | 327 to 405 |
| 326 to 409 | 325 to 420 | 323 to 418 | 327 to 406 |
| 326 to 410 | 325 to 421 | 323 to 419 | 327 to 407 |
| 326 to 411 | 325 to 422 | 323 to 420 | 327 to 408 |
| 326 to 412 | 325 to 423 | 323 to 421 | 327 to 409 |
| 326 to 413 | 325 to 424 | 323 to 422 | 327 to 410 |
| 326 to 414 | 325 to 425 | 323 to 423 | 327 to 411 |
| 326 to 415 | 325 to 426 | 323 to 424 | 327 to 412 |
| 326 to 416 | 325 to 427 | 323 to 425 | 327 to 413 |
| 326 to 417 | 325 to 428 | 323 to 426 | 327 to 414 |
| 326 to 418 | 325 to 429 | 323 to 427 | 327 to 415 |
| 326 to 419 | 325 to 430 | 323 to 428 | 327 to 416 |
| 326 to 420 | 325 to 431 | 323 to 429 | 327 to 417 |
| 326 to 421 | 325 to 432 | 323 to 430 | 327 to 418 |
| 326 to 422 | 325 to 433 | 323 to 431 | 327 to 419 |
| 326 to 423 | 325 to 434 | 323 to 432 | 327 to 420 |
| 326 to 424 | 325 to 435 | 323 to 433 | 327 to 421 |
| 326 to 425 | 325 to 436 | 323 to 434 | 327 to 422 |
| 326 to 426 | 325 to 437 | 323 to 435 | 327 to 423 |
| 326 to 427 | 325 to 438 | 323 to 436 | 327 to 424 |
| 326 to 428 | 325 to 439 | 323 to 437 | 327 to 425 |
| 326 to 429 | 325 to 440 | 323 to 438 | 327 to 426 |
| 326 to 430 | 326 to 404 | 324 to 402 | 327 to 427 |
| 326 to 431 | 326 to 405 | 324 to 403 | 327 to 428 |
| 326 to 432 | 326 to 406 | 324 to 404 | 327 to 429 |
| 326 to 433 | 326 to 407 | 324 to 405 | 327 to 430 |
| 326 to 434 | 326 to 408 | 324 to 406 | 327 to 431 |
| 326 to 435 | 326 to 409 | 324 to 407 | 327 to 432 |
| 326 to 436 | 326 to 410 | 324 to 408 | 327 to 433 |
| 326 to 437 | 326 to 411 | 324 to 409 | 327 to 434 |
| 326 to 438 | 326 to 412 | 324 to 410 | 327 to 435 |
| 326 to 439 | 326 to 413 | 324 to 411 | 327 to 436 |
| 326 to 440 | 326 to 414 | 324 to 412 | 327 to 437 |
| 326 to 441 | 326 to 415 | 324 to 413 | 328 to 403 |
| 326 to 442 | 326 to 416 | 324 to 414 | 328 to 404 |
| 326 to 443 | 326 to 417 | 324 to 415 | 328 to 405 |
| 326 to 444 | 326 to 418 | 324 to 416 | 328 to 406 |
| 326 to 445 | 326 to 419 | 324 to 417 | 328 to 407 |
| 326 to 446 | 326 to 420 | 324 to 418 | 328 to 408 |
| 326 to 447 | 326 to 421 | 324 to 419 | 328 to 409 |
| 327 to 406 | 326 to 422 | 324 to 420 | 328 to 410 |
| 327 to 407 | 326 to 423 | 324 to 421 | 328 to 411 |
| 327 to 408 | 326 to 424 | 324 to 422 | 328 to 412 |
| 327 to 409 | 326 to 425 | 324 to 423 | 328 to 413 |
| 327 to 410 | 326 to 426 | 324 to 424 | 328 to 414 |
| 327 to 411 | 326 to 427 | 324 to 425 | 328 to 415 |
| 327 to 412 | 326 to 428 | 324 to 426 | 328 to 416 |
| 327 to 413 | 326 to 429 | 324 to 427 | 328 to 417 |
| 327 to 414 | 326 to 430 | 324 to 428 | 328 to 418 |
| 327 to 415 | 326 to 431 | 324 to 429 | 328 to 419 |
| 327 to 416 | 326 to 432 | 324 to 430 | 328 to 420 |
| 327 to 417 | 326 to 433 | 324 to 431 | 328 to 421 |
| 327 to 418 | 326 to 434 | 324 to 432 | 328 to 422 |
| 327 to 419 | 326 to 435 | 324 to 433 | 328 to 423 |
| 327 to 420 | 326 to 436 | 324 to 434 | 328 to 424 |
| 327 to 421 | 326 to 437 | 324 to 435 | 328 to 425 |
| 327 to 422 | 326 to 438 | 324 to 436 | 328 to 426 |
| 327 to 423 | 326 to 439 | 324 to 437 | 328 to 427 |
| 327 to 424 | 326 to 440 | 324 to 438 | 328 to 428 |
| 327 to 425 | 327 to 404 | 325 to 402 | 328 to 429 |
| 327 to 426 | 327 to 405 | 325 to 403 | 328 to 430 |
| 327 to 427 | 327 to 406 | 325 to 404 | 328 to 431 |
| 327 to 428 | 327 to 407 | 325 to 405 | 328 to 432 |
| 327 to 429 | 327 to 408 | 325 to 406 | 328 to 433 |
| 327 to 430 | 327 to 409 | 325 to 407 | 328 to 434 |
| 327 to 431 | 327 to 410 | 325 to 408 | 328 to 435 |
| 327 to 432 | 327 to 411 | 325 to 409 | 328 to 436 |
| 327 to 433 | 327 to 412 | 325 to 410 | 328 to 437 |
| 327 to 434 | 327 to 413 | 325 to 411 | 329 to 403 |
| 327 to 435 | 327 to 414 | 325 to 412 | 329 to 404 |
| 327 to 436 | 327 to 415 | 325 to 413 | 329 to 405 |
| 327 to 437 | 327 to 416 | 325 to 414 | 329 to 406 |
| 327 to 438 | 327 to 417 | 325 to 415 | 329 to 407 |
| 327 to 439 | 327 to 418 | 325 to 416 | 329 to 408 |
| 327 to 440 | 327 to 419 | 325 to 417 | 329 to 409 |
| 327 to 441 | 327 to 420 | 325 to 418 | 329 to 410 |
| 327 to 442 | 327 to 421 | 325 to 419 | 329 to 411 |
| 327 to 443 | 327 to 422 | 325 to 420 | 329 to 412 |
| 327 to 444 | 327 to 423 | 325 to 421 | 329 to 413 |
| 327 to 445 | 327 to 424 | 325 to 422 | 329 to 414 |
| 327 to 446 | 327 to 425 | 325 to 423 | 329 to 415 |
| 327 to 447 | 327 to 426 | 325 to 424 | 329 to 416 |
| 328 to 406 | 327 to 427 | 325 to 425 | 329 to 417 |
| 328 to 407 | 327 to 428 | 325 to 426 | 329 to 418 |
| 328 to 408 | 327 to 429 | 325 to 427 | 329 to 419 |
| 328 to 409 | 327 to 430 | 325 to 428 | 329 to 420 |
| 328 to 410 | 327 to 431 | 325 to 429 | 329 to 421 |
| 328 to 411 | 327 to 432 | 325 to 430 | 329 to 422 |
| 328 to 412 | 327 to 433 | 325 to 431 | 329 to 423 |
| 328 to 413 | 327 to 434 | 325 to 432 | 329 to 424 |
| 328 to 414 | 327 to 435 | 325 to 433 | 329 to 425 |
| 328 to 415 | 327 to 436 | 325 to 434 | 329 to 426 |
| 328 to 416 | 327 to 437 | 325 to 435 | 329 to 427 |
| 328 to 417 | 327 to 438 | 325 to 436 | 329 to 428 |
| 328 to 418 | 327 to 439 | 325 to 437 | 329 to 429 |
| 328 to 419 | 327 to 440 | 325 to 438 | 329 to 430 |
| 328 to 420 | 328 to 404 | 326 to 402 | 329 to 431 |
| 328 to 421 | 328 to 405 | 326 to 403 | 329 to 432 |
| 328 to 422 | 328 to 406 | 326 to 404 | 329 to 433 |
| 328 to 423 | 328 to 407 | 326 to 405 | 329 to 434 |
| 328 to 424 | 328 to 408 | 326 to 406 | 329 to 435 |
| 328 to 425 | 328 to 409 | 326 to 407 | 329 to 436 |
| 328 to 426 | 328 to 410 | 326 to 408 | 329 to 437 |
| 328 to 427 | 328 to 411 | 326 to 409 | 330 to 403 |
| 328 to 428 | 328 to 412 | 326 to 410 | 330 to 404 |
| 328 to 429 | 328 to 413 | 326 to 411 | 330 to 405 |
| 328 to 430 | 328 to 414 | 326 to 412 | 330 to 406 |
| 328 to 431 | 328 to 415 | 326 to 413 | 330 to 407 |
| 328 to 432 | 328 to 416 | 326 to 414 | 330 to 408 |
| 328 to 433 | 328 to 417 | 326 to 415 | 330 to 409 |
| 328 to 434 | 328 to 418 | 326 to 416 | 330 to 410 |
| 328 to 435 | 328 to 419 | 326 to 417 | 330 to 411 |
| 328 to 436 | 328 to 420 | 326 to 418 | 330 to 412 |
| 328 to 437 | 328 to 421 | 326 to 419 | 330 to 413 |
| 328 to 438 | 328 to 422 | 326 to 420 | 330 to 414 |
| 328 to 439 | 328 to 423 | 326 to 421 | 330 to 415 |
| 328 to 440 | 328 to 424 | 326 to 422 | 330 to 416 |
| 328 to 441 | 328 to 425 | 326 to 423 | 330 to 417 |
| 328 to 442 | 328 to 426 | 326 to 424 | 330 to 418 |
| 328 to 443 | 328 to 427 | 326 to 425 | 330 to 419 |
| 328 to 444 | 328 to 428 | 326 to 426 | 330 to 420 |
| 328 to 445 | 328 to 429 | 326 to 427 | 330 to 421 |
| 328 to 446 | 328 to 430 | 326 to 428 | 330 to 422 |
| 328 to 447 | 328 to 431 | 326 to 429 | 330 to 423 |
| 329 to 406 | 328 to 432 | 326 to 430 | 330 to 424 |
| 329 to 407 | 328 to 433 | 326 to 431 | 330 to 425 |
| 329 to 408 | 328 to 434 | 326 to 432 | 330 to 426 |
| 329 to 409 | 328 to 435 | 326 to 433 | 330 to 427 |
| 329 to 410 | 328 to 436 | 326 to 434 | 330 to 428 |
| 329 to 411 | 328 to 437 | 326 to 435 | 330 to 429 |
| 329 to 412 | 328 to 438 | 326 to 436 | 330 to 430 |
| 329 to 413 | 328 to 439 | 326 to 437 | 330 to 431 |
| 329 to 414 | 328 to 440 | 326 to 438 | 330 to 432 |
| 329 to 415 | 329 to 404 | 327 to 402 | 330 to 433 |
| 329 to 416 | 329 to 405 | 327 to 403 | 330 to 434 |
| 329 to 417 | 329 to 406 | 327 to 404 | 330 to 435 |
| 329 to 418 | 329 to 407 | 327 to 405 | 330 to 436 |
| 329 to 419 | 329 to 408 | 327 to 406 | 330 to 437 |
| 329 to 420 | 329 to 409 | 327 to 407 | 331 to 403 |
| 329 to 421 | 329 to 410 | 327 to 408 | 331 to 404 |
| 329 to 422 | 329 to 411 | 327 to 409 | 331 to 405 |
| 329 to 423 | 329 to 412 | 327 to 410 | 331 to 406 |
| 329 to 424 | 329 to 413 | 327 to 411 | 331 to 407 |
| 329 to 425 | 329 to 414 | 327 to 412 | 331 to 408 |
| 329 to 426 | 329 to 415 | 327 to 413 | 331 to 409 |
| 329 to 427 | 329 to 416 | 327 to 414 | 331 to 410 |
| 329 to 428 | 329 to 417 | 327 to 415 | 331 to 411 |
| 329 to 429 | 329 to 418 | 327 to 416 | 331 to 412 |
| 329 to 430 | 329 to 419 | 327 to 417 | 331 to 413 |
| 329 to 431 | 329 to 420 | 327 to 418 | 331 to 414 |
| 329 to 432 | 329 to 421 | 327 to 419 | 331 to 415 |
| 329 to 433 | 329 to 422 | 327 to 420 | 331 to 416 |
| 329 to 434 | 329 to 423 | 327 to 421 | 331 to 417 |
| 329 to 435 | 329 to 424 | 327 to 422 | 331 to 418 |
| 329 to 436 | 329 to 425 | 327 to 423 | 331 to 419 |
| 329 to 437 | 329 to 426 | 327 to 424 | 331 to 420 |
| 329 to 438 | 329 to 427 | 327 to 425 | 331 to 421 |
| 329 to 439 | 329 to 428 | 327 to 426 | 331 to 422 |
| 329 to 440 | 329 to 429 | 327 to 427 | 331 to 423 |
| 329 to 441 | 329 to 430 | 327 to 428 | 331 to 424 |
| 329 to 442 | 329 to 431 | 327 to 429 | 331 to 425 |
| 329 to 443 | 329 to 432 | 327 to 430 | 331 to 426 |
| 329 to 444 | 329 to 433 | 327 to 431 | 331 to 427 |
| 329 to 445 | 329 to 434 | 327 to 432 | 331 to 428 |
| 329 to 446 | 329 to 435 | 327 to 433 | 331 to 429 |
| 329 to 447 | 329 to 436 | 327 to 434 | 331 to 430 |
| 330 to 406 | 329 to 437 | 327 to 435 | 331 to 431 |
| 330 to 407 | 329 to 438 | 327 to 436 | 331 to 432 |
| 330 to 408 | 329 to 439 | 327 to 437 | 331 to 433 |
| 330 to 409 | 329 to 440 | 327 to 438 | 331 to 434 |
| 330 to 410 | 330 to 404 | 328 to 402 | 331 to 435 |
| 330 to 411 | 330 to 405 | 328 to 403 | 331 to 436 |
| 330 to 412 | 330 to 406 | 328 to 404 | 331 to 437 |
| 330 to 413 | 330 to 407 | 328 to 405 | 332 to 403 |
| 330 to 414 | 330 to 408 | 328 to 406 | 332 to 404 |
| 330 to 415 | 330 to 409 | 328 to 407 | 332 to 405 |
| 330 to 416 | 330 to 410 | 328 to 408 | 332 to 406 |
| 330 to 417 | 330 to 411 | 328 to 409 | 332 to 407 |
| 330 to 418 | 330 to 412 | 328 to 410 | 332 to 408 |
| 330 to 419 | 330 to 413 | 328 to 411 | 332 to 409 |
| 330 to 420 | 330 to 414 | 328 to 412 | 332 to 410 |
| 330 to 421 | 330 to 415 | 328 to 413 | 332 to 411 |
| 330 to 422 | 330 to 416 | 328 to 414 | 332 to 412 |
| 330 to 423 | 330 to 417 | 328 to 415 | 332 to 413 |
| 330 to 424 | 330 to 418 | 328 to 416 | 332 to 414 |
| 330 to 425 | 330 to 419 | 328 to 417 | 332 to 415 |
| 330 to 426 | 330 to 420 | 328 to 418 | 332 to 416 |
| 330 to 427 | 330 to 421 | 328 to 419 | 332 to 417 |
| 330 to 428 | 330 to 422 | 328 to 420 | 332 to 418 |
| 330 to 429 | 330 to 423 | 328 to 421 | 332 to 419 |
| 330 to 430 | 330 to 424 | 328 to 422 | 332 to 420 |
| 330 to 431 | 330 to 425 | 328 to 423 | 332 to 421 |
| 330 to 432 | 330 to 426 | 328 to 424 | 332 to 422 |
| 330 to 433 | 330 to 427 | 328 to 425 | 332 to 423 |
| 330 to 434 | 330 to 428 | 328 to 426 | 332 to 424 |
| 330 to 435 | 330 to 429 | 328 to 427 | 332 to 425 |
| 330 to 436 | 330 to 430 | 328 to 428 | 332 to 426 |
| 330 to 437 | 330 to 431 | 328 to 429 | 332 to 427 |
| 330 to 438 | 330 to 432 | 328 to 430 | 332 to 428 |
| 330 to 439 | 330 to 433 | 328 to 431 | 332 to 429 |
| 330 to 440 | 330 to 434 | 328 to 432 | 332 to 430 |
| 330 to 441 | 330 to 435 | 328 to 433 | 332 to 431 |
| 330 to 442 | 330 to 436 | 328 to 434 | 332 to 432 |
| 330 to 443 | 330 to 437 | 328 to 435 | 332 to 433 |
| 330 to 444 | 330 to 438 | 328 to 436 | 332 to 434 |
| 330 to 445 | 330 to 439 | 328 to 437 | 332 to 435 |
| 330 to 446 | 330 to 440 | 328 to 438 | 332 to 436 |
| 330 to 447 | 331 to 404 | 329 to 402 | 332 to 437 |
| 331 to 406 | 331 to 405 | 329 to 403 | 333 to 403 |
| 331 to 407 | 331 to 406 | 329 to 404 | 333 to 404 |
| 331 to 408 | 331 to 407 | 329 to 405 | 333 to 405 |
| 331 to 409 | 331 to 408 | 329 to 406 | 333 to 406 |
| 331 to 410 | 331 to 409 | 329 to 407 | 333 to 407 |
| 331 to 411 | 331 to 410 | 329 to 408 | 333 to 408 |
| 331 to 412 | 331 to 411 | 329 to 409 | 333 to 409 |
| 331 to 413 | 331 to 412 | 329 to 410 | 333 to 410 |
| 331 to 414 | 331 to 413 | 329 to 411 | 333 to 411 |
| 331 to 415 | 331 to 414 | 329 to 412 | 333 to 412 |
| 331 to 416 | 331 to 415 | 329 to 413 | 333 to 413 |
| 331 to 417 | 331 to 416 | 329 to 414 | 333 to 414 |
| 331 to 418 | 331 to 417 | 329 to 415 | 333 to 415 |
| 331 to 419 | 331 to 418 | 329 to 416 | 333 to 416 |
| 331 to 420 | 331 to 419 | 329 to 417 | 333 to 417 |
| 331 to 421 | 331 to 420 | 329 to 418 | 333 to 418 |
| 331 to 422 | 331 to 421 | 329 to 419 | 333 to 419 |
| 331 to 423 | 331 to 422 | 329 to 420 | 333 to 420 |
| 331 to 424 | 331 to 423 | 329 to 421 | 333 to 421 |
| 331 to 425 | 331 to 424 | 329 to 422 | 333 to 422 |
| 331 to 426 | 331 to 425 | 329 to 423 | 333 to 423 |
| 331 to 427 | 331 to 426 | 329 to 424 | 333 to 424 |
| 331 to 428 | 331 to 427 | 329 to 425 | 333 to 425 |
| 331 to 429 | 331 to 428 | 329 to 426 | 333 to 426 |
| 331 to 430 | 331 to 429 | 329 to 427 | 333 to 427 |
| 331 to 431 | 331 to 430 | 329 to 428 | 333 to 428 |
| 331 to 432 | 331 to 431 | 329 to 429 | 333 to 429 |
| 331 to 433 | 331 to 432 | 329 to 430 | 333 to 430 |
| 331 to 434 | 331 to 433 | 329 to 431 | 333 to 431 |
| 331 to 435 | 331 to 434 | 329 to 432 | 333 to 432 |
| 331 to 436 | 331 to 435 | 329 to 433 | 333 to 433 |
| 331 to 437 | 331 to 436 | 329 to 434 | 333 to 434 |
| 331 to 438 | 331 to 437 | 329 to 435 | 333 to 435 |
| 331 to 439 | 331 to 438 | 329 to 436 | 333 to 436 |
| 331 to 440 | 331 to 439 | 329 to 437 | 333 to 437 |
| 331 to 441 | 331 to 440 | 329 to 438 | 334 to 403 |
| 331 to 442 | 332 to 404 | 330 to 402 | 334 to 404 |
| 331 to 443 | 332 to 405 | 330 to 403 | 334 to 405 |
| 331 to 444 | 332 to 406 | 330 to 404 | 334 to 406 |
| 331 to 445 | 332 to 407 | 330 to 405 | 334 to 407 |
| 331 to 446 | 332 to 408 | 330 to 406 | 334 to 408 |
| 331 to 447 | 332 to 409 | 330 to 407 | 334 to 409 |
| 332 to 406 | 332 to 410 | 330 to 408 | 334 to 410 |
| 332 to 407 | 332 to 411 | 330 to 409 | 334 to 411 |
| 332 to 408 | 332 to 412 | 330 to 410 | 334 to 412 |
| 332 to 409 | 332 to 413 | 330 to 411 | 334 to 413 |
| 332 to 410 | 332 to 414 | 330 to 412 | 334 to 414 |
| 332 to 411 | 332 to 415 | 330 to 413 | 334 to 415 |
| 332 to 412 | 332 to 416 | 330 to 414 | 334 to 416 |
| 332 to 413 | 332 to 417 | 330 to 415 | 334 to 417 |
| 332 to 414 | 332 to 418 | 330 to 416 | 334 to 418 |
| 332 to 415 | 332 to 419 | 330 to 417 | 334 to 419 |
| 332 to 416 | 332 to 420 | 330 to 418 | 334 to 420 |
| 332 to 417 | 332 to 421 | 330 to 419 | 334 to 421 |
| 332 to 418 | 332 to 422 | 330 to 420 | 334 to 422 |
| 332 to 419 | 332 to 423 | 330 to 421 | 334 to 423 |
| 332 to 420 | 332 to 424 | 330 to 422 | 334 to 424 |
| 332 to 421 | 332 to 425 | 330 to 423 | 334 to 425 |
| 332 to 422 | 332 to 426 | 330 to 424 | 334 to 426 |
| 332 to 423 | 332 to 427 | 330 to 425 | 334 to 427 |
| 332 to 424 | 332 to 428 | 330 to 426 | 334 to 428 |
| 332 to 425 | 332 to 429 | 330 to 427 | 334 to 429 |
| 332 to 426 | 332 to 430 | 330 to 428 | 334 to 430 |
| 332 to 427 | 332 to 431 | 330 to 429 | 334 to 431 |
| 332 to 428 | 332 to 432 | 330 to 430 | 334 to 432 |
| 332 to 429 | 332 to 433 | 330 to 431 | 334 to 433 |
| 332 to 430 | 332 to 434 | 330 to 432 | 334 to 434 |
| 332 to 431 | 332 to 435 | 330 to 433 | 334 to 435 |
| 332 to 432 | 332 to 436 | 330 to 434 | 334 to 436 |
| 332 to 433 | 332 to 437 | 330 to 435 | 334 to 437 |
| 332 to 434 | 332 to 438 | 330 to 436 | 335 to 403 |
| 332 to 435 | 332 to 439 | 330 to 437 | 335 to 404 |
| 332 to 436 | 332 to 440 | 330 to 438 | 335 to 405 |
| 332 to 437 | 333 to 404 | 331 to 402 | 335 to 406 |
| 332 to 438 | 333 to 405 | 331 to 403 | 335 to 407 |
| 332 to 439 | 333 to 406 | 331 to 404 | 335 to 408 |
| 332 to 440 | 333 to 407 | 331 to 405 | 335 to 409 |
| 332 to 441 | 333 to 408 | 331 to 406 | 335 to 410 |
| 332 to 442 | 333 to 409 | 331 to 407 | 335 to 411 |
| 332 to 443 | 333 to 410 | 331 to 408 | 335 to 412 |
| 332 to 444 | 333 to 411 | 331 to 409 | 335 to 413 |
| 332 to 445 | 333 to 412 | 331 to 410 | 335 to 414 |
| 332 to 446 | 333 to 413 | 331 to 411 | 335 to 415 |
| 332 to 447 | 333 to 414 | 331 to 412 | 335 to 416 |
| 333 to 406 | 333 to 415 | 331 to 413 | 335 to 417 |
| 333 to 407 | 333 to 416 | 331 to 414 | 335 to 418 |
| 333 to 408 | 333 to 417 | 331 to 415 | 335 to 419 |
| 333 to 409 | 333 to 418 | 331 to 416 | 335 to 420 |
| 333 to 410 | 333 to 419 | 331 to 417 | 335 to 421 |
| 333 to 411 | 333 to 420 | 331 to 418 | 335 to 422 |
| 333 to 412 | 333 to 421 | 331 to 419 | 335 to 423 |
| 333 to 413 | 333 to 422 | 331 to 420 | 335 to 424 |
| 333 to 414 | 333 to 423 | 331 to 421 | 335 to 425 |
| 333 to 415 | 333 to 424 | 331 to 422 | 335 to 426 |
| 333 to 416 | 333 to 425 | 331 to 423 | 335 to 427 |
| 333 to 417 | 333 to 426 | 331 to 424 | 335 to 428 |
| 333 to 418 | 333 to 427 | 331 to 425 | 335 to 429 |
| 333 to 419 | 333 to 428 | 331 to 426 | 335 to 430 |
| 333 to 420 | 333 to 429 | 331 to 427 | 335 to 431 |
| 333 to 421 | 333 to 430 | 331 to 428 | 335 to 432 |
| 333 to 422 | 333 to 431 | 331 to 429 | 335 to 433 |
| 333 to 423 | 333 to 432 | 331 to 430 | 335 to 434 |
| 333 to 424 | 333 to 433 | 331 to 431 | 335 to 435 |
| 333 to 425 | 333 to 434 | 331 to 432 | 335 to 436 |
| 333 to 426 | 333 to 435 | 331 to 433 | 335 to 437 |
| 333 to 427 | 333 to 436 | 331 to 434 | 336 to 403 |
| 333 to 428 | 333 to 437 | 331 to 435 | 336 to 404 |
| 333 to 429 | 333 to 438 | 331 to 436 | 336 to 405 |
| 333 to 430 | 333 to 439 | 331 to 437 | 336 to 406 |
| 333 to 431 | 333 to 440 | 331 to 438 | 336 to 407 |
| 333 to 432 | 334 to 404 | 332 to 402 | 336 to 408 |
| 333 to 433 | 334 to 405 | 332 to 403 | 336 to 409 |
| 333 to 434 | 334 to 406 | 332 to 404 | 336 to 410 |
| 333 to 435 | 334 to 407 | 332 to 405 | 336 to 411 |
| 333 to 436 | 334 to 408 | 332 to 406 | 336 to 412 |
| 333 to 437 | 334 to 409 | 332 to 407 | 336 to 413 |
| 333 to 438 | 334 to 410 | 332 to 408 | 336 to 414 |
| 333 to 439 | 334 to 411 | 332 to 409 | 336 to 415 |
| 333 to 440 | 334 to 412 | 332 to 410 | 336 to 416 |
| 333 to 441 | 334 to 413 | 332 to 411 | 336 to 417 |
| 333 to 442 | 334 to 414 | 332 to 412 | 336 to 418 |
| 333 to 443 | 334 to 415 | 332 to 413 | 336 to 419 |
| 333 to 444 | 334 to 416 | 332 to 414 | 336 to 420 |
| 333 to 445 | 334 to 417 | 332 to 415 | 336 to 421 |
| 333 to 446 | 334 to 418 | 332 to 416 | 336 to 422 |
| 333 to 447 | 334 to 419 | 332 to 417 | 336 to 423 |
| 334 to 406 | 334 to 420 | 332 to 418 | 336 to 424 |
| 334 to 407 | 334 to 421 | 332 to 419 | 336 to 425 |
| 334 to 408 | 334 to 422 | 332 to 420 | 336 to 426 |
| 334 to 409 | 334 to 423 | 332 to 421 | 336 to 427 |
| 334 to 410 | 334 to 424 | 332 to 422 | 336 to 428 |
| 334 to 411 | 334 to 425 | 332 to 423 | 336 to 429 |
| 334 to 412 | 334 to 426 | 332 to 424 | 336 to 430 |
| 334 to 413 | 334 to 427 | 332 to 425 | 336 to 431 |
| 334 to 414 | 334 to 428 | 332 to 426 | 336 to 432 |
| 334 to 415 | 334 to 429 | 332 to 427 | 336 to 433 |
| 334 to 416 | 334 to 430 | 332 to 428 | 336 to 434 |
| 334 to 417 | 334 to 431 | 332 to 429 | 336 to 435 |
| 334 to 418 | 334 to 432 | 332 to 430 | 336 to 436 |
| 334 to 419 | 334 to 433 | 332 to 431 | 336 to 437 |
| 334 to 420 | 334 to 434 | 332 to 432 | 337 to 403 |
| 334 to 421 | 334 to 435 | 332 to 433 | 337 to 404 |
| 334 to 422 | 334 to 436 | 332 to 434 | 337 to 405 |
| 334 to 423 | 334 to 437 | 332 to 435 | 337 to 406 |
| 334 to 424 | 334 to 438 | 332 to 436 | 337 to 407 |
| 334 to 425 | 334 to 439 | 332 to 437 | 337 to 408 |
| 334 to 426 | 334 to 440 | 332 to 438 | 337 to 409 |
| 334 to 427 | 335 to 404 | 333 to 402 | 337 to 410 |
| 334 to 428 | 335 to 405 | 333 to 403 | 337 to 411 |
| 334 to 429 | 335 to 406 | 333 to 404 | 337 to 412 |
| 334 to 430 | 335 to 407 | 333 to 405 | 337 to 413 |
| 334 to 431 | 335 to 408 | 333 to 406 | 337 to 414 |
| 334 to 432 | 335 to 409 | 333 to 407 | 337 to 415 |
| 334 to 433 | 335 to 410 | 333 to 408 | 337 to 416 |
| 334 to 434 | 335 to 411 | 333 to 409 | 337 to 417 |
| 334 to 435 | 335 to 412 | 333 to 410 | 337 to 418 |
| 334 to 436 | 335 to 413 | 333 to 411 | 337 to 419 |
| 334 to 437 | 335 to 414 | 333 to 412 | 337 to 420 |
| 334 to 438 | 335 to 415 | 333 to 413 | 337 to 421 |
| 334 to 439 | 335 to 416 | 333 to 414 | 337 to 422 |
| 334 to 440 | 335 to 417 | 333 to 415 | 337 to 423 |
| 334 to 441 | 335 to 418 | 333 to 416 | 337 to 424 |
| 334 to 442 | 335 to 419 | 333 to 417 | 337 to 425 |
| 334 to 443 | 335 to 420 | 333 to 418 | 337 to 426 |
| 334 to 444 | 335 to 421 | 333 to 419 | 337 to 427 |
| 334 to 445 | 335 to 422 | 333 to 420 | 337 to 428 |
| 334 to 446 | 335 to 423 | 333 to 421 | 337 to 429 |
| 334 to 447 | 335 to 424 | 333 to 422 | 337 to 430 |
| 335 to 406 | 335 to 425 | 333 to 423 | 337 to 431 |
| 335 to 407 | 335 to 426 | 333 to 424 | 337 to 432 |
| 335 to 408 | 335 to 427 | 333 to 425 | 337 to 433 |
| 335 to 409 | 335 to 428 | 333 to 426 | 337 to 434 |
| 335 to 410 | 335 to 429 | 333 to 427 | 337 to 435 |
| 335 to 411 | 335 to 430 | 333 to 428 | 337 to 436 |
| 335 to 412 | 335 to 431 | 333 to 429 | 337 to 437 |
| 335 to 413 | 335 to 432 | 333 to 430 | 338 to 403 |
| 335 to 414 | 335 to 433 | 333 to 431 | 338 to 404 |
| 335 to 415 | 335 to 434 | 333 to 432 | 338 to 405 |
| 335 to 416 | 335 to 435 | 333 to 433 | 338 to 406 |
| 335 to 417 | 335 to 436 | 333 to 434 | 338 to 407 |
| 335 to 418 | 335 to 437 | 333 to 435 | 338 to 408 |
| 335 to 419 | 335 to 438 | 333 to 436 | 338 to 409 |
| 335 to 420 | 335 to 439 | 333 to 437 | 338 to 410 |
| 335 to 421 | 335 to 440 | 333 to 438 | 338 to 411 |
| 335 to 422 | 336 to 404 | 334 to 402 | 338 to 412 |
| 335 to 423 | 336 to 405 | 334 to 403 | 338 to 413 |
| 335 to 424 | 336 to 406 | 334 to 404 | 338 to 414 |
| 335 to 425 | 336 to 407 | 334 to 405 | 338 to 415 |
| 335 to 426 | 336 to 408 | 334 to 406 | 338 to 416 |
| 335 to 427 | 336 to 409 | 334 to 407 | 338 to 417 |
| 335 to 428 | 336 to 410 | 334 to 408 | 338 to 418 |
| 335 to 429 | 336 to 411 | 334 to 409 | 338 to 419 |
| 335 to 430 | 336 to 412 | 334 to 410 | 338 to 420 |
| 335 to 431 | 336 to 413 | 334 to 411 | 338 to 421 |
| 335 to 432 | 336 to 414 | 334 to 412 | 338 to 422 |
| 335 to 433 | 336 to 415 | 334 to 413 | 338 to 423 |
| 335 to 434 | 336 to 416 | 334 to 414 | 338 to 424 |
| 335 to 435 | 336 to 417 | 334 to 415 | 338 to 425 |
| 335 to 436 | 336 to 418 | 334 to 416 | 338 to 426 |
| 335 to 437 | 336 to 419 | 334 to 417 | 338 to 427 |
| 335 to 438 | 336 to 420 | 334 to 418 | 338 to 428 |
| 335 to 439 | 336 to 421 | 334 to 419 | 338 to 429 |
| 335 to 440 | 336 to 422 | 334 to 420 | 338 to 430 |
| 335 to 441 | 336 to 423 | 334 to 421 | 338 to 431 |
| 335 to 442 | 336 to 424 | 334 to 422 | 338 to 432 |
| 335 to 443 | 336 to 425 | 334 to 423 | 338 to 433 |
| 335 to 444 | 336 to 426 | 334 to 424 | 338 to 434 |
| 335 to 445 | 336 to 427 | 334 to 425 | 338 to 435 |
| 335 to 446 | 336 to 428 | 334 to 426 | 338 to 436 |
| 335 to 447 | 336 to 429 | 334 to 427 | 338 to 437 |
| 336 to 406 | 336 to 430 | 334 to 428 | 339 to 403 |
| 336 to 407 | 336 to 431 | 334 to 429 | 339 to 404 |
| 336 to 408 | 336 to 432 | 334 to 430 | 339 to 405 |
| 336 to 409 | 336 to 433 | 334 to 431 | 339 to 406 |
| 336 to 410 | 336 to 434 | 334 to 432 | 339 to 407 |
| 336 to 411 | 336 to 435 | 334 to 433 | 339 to 408 |
| 336 to 412 | 336 to 436 | 334 to 434 | 339 to 409 |
| 336 to 413 | 336 to 437 | 334 to 435 | 339 to 410 |
| 336 to 414 | 336 to 438 | 334 to 436 | 339 to 411 |
| 336 to 415 | 336 to 439 | 334 to 437 | 339 to 412 |
| 336 to 416 | 336 to 440 | 334 to 438 | 339 to 413 |
| 336 to 417 | 337 to 404 | 335 to 402 | 339 to 414 |
| 336 to 418 | 337 to 405 | 335 to 403 | 339 to 415 |
| 336 to 419 | 337 to 406 | 335 to 404 | 339 to 416 |
| 336 to 420 | 337 to 407 | 335 to 405 | 339 to 417 |
| 336 to 421 | 337 to 408 | 335 to 406 | 339 to 418 |
| 336 to 422 | 337 to 409 | 335 to 407 | 339 to 419 |
| 336 to 423 | 337 to 410 | 335 to 408 | 339 to 420 |
| 336 to 424 | 337 to 411 | 335 to 409 | 339 to 421 |
| 336 to 425 | 337 to 412 | 335 to 410 | 339 to 422 |
| 336 to 426 | 337 to 413 | 335 to 411 | 339 to 423 |
| 336 to 427 | 337 to 414 | 335 to 412 | 339 to 424 |
| 336 to 428 | 337 to 415 | 335 to 413 | 339 to 425 |
| 336 to 429 | 337 to 416 | 335 to 414 | 339 to 426 |
| 336 to 430 | 337 to 417 | 335 to 415 | 339 to 427 |
| 336 to 431 | 337 to 418 | 335 to 416 | 339 to 428 |
| 336 to 432 | 337 to 419 | 335 to 417 | 339 to 429 |
| 336 to 433 | 337 to 420 | 335 to 418 | 339 to 430 |
| 336 to 434 | 337 to 421 | 335 to 419 | 339 to 431 |
| 336 to 435 | 337 to 422 | 335 to 420 | 339 to 432 |
| 336 to 436 | 337 to 423 | 335 to 421 | 339 to 433 |
| 336 to 437 | 337 to 424 | 335 to 422 | 339 to 434 |
| 336 to 438 | 337 to 425 | 335 to 423 | 339 to 435 |
| 336 to 439 | 337 to 426 | 335 to 424 | 339 to 436 |
| 336 to 440 | 337 to 427 | 335 to 425 | 339 to 437 |
| 336 to 441 | 337 to 428 | 335 to 426 | 340 to 403 |
| 336 to 442 | 337 to 429 | 335 to 427 | 340 to 404 |
| 336 to 443 | 337 to 430 | 335 to 428 | 340 to 405 |
| 336 to 444 | 337 to 431 | 335 to 429 | 340 to 406 |
| 336 to 445 | 337 to 432 | 335 to 430 | 340 to 407 |
| 336 to 446 | 337 to 433 | 335 to 431 | 340 to 408 |
| 336 to 447 | 337 to 434 | 335 to 432 | 340 to 409 |
| 337 to 406 | 337 to 435 | 335 to 433 | 340 to 410 |
| 337 to 407 | 337 to 436 | 335 to 434 | 340 to 411 |
| 337 to 408 | 337 to 437 | 335 to 435 | 340 to 412 |
| 337 to 409 | 337 to 438 | 335 to 436 | 340 to 413 |
| 337 to 410 | 337 to 439 | 335 to 437 | 340 to 414 |
| 337 to 411 | 337 to 440 | 335 to 438 | 340 to 415 |
| 337 to 412 | 338 to 404 | 336 to 402 | 340 to 416 |
| 337 to 413 | 338 to 405 | 336 to 403 | 340 to 417 |
| 337 to 414 | 338 to 406 | 336 to 404 | 340 to 418 |
| 337 to 415 | 338 to 407 | 336 to 405 | 340 to 419 |
| 337 to 416 | 338 to 408 | 336 to 406 | 340 to 420 |
| 337 to 417 | 338 to 409 | 336 to 407 | 340 to 421 |
| 337 to 418 | 338 to 410 | 336 to 408 | 340 to 422 |
| 337 to 419 | 338 to 411 | 336 to 409 | 340 to 423 |
| 337 to 420 | 338 to 412 | 336 to 410 | 340 to 424 |
| 337 to 421 | 338 to 413 | 336 to 411 | 340 to 425 |
| 337 to 422 | 338 to 414 | 336 to 412 | 340 to 426 |
| 337 to 423 | 338 to 415 | 336 to 413 | 340 to 427 |
| 337 to 424 | 338 to 416 | 336 to 414 | 340 to 428 |
| 337 to 425 | 338 to 417 | 336 to 415 | 340 to 429 |
| 337 to 426 | 338 to 418 | 336 to 416 | 340 to 430 |
| 337 to 427 | 338 to 419 | 336 to 417 | 340 to 431 |
| 337 to 428 | 338 to 420 | 336 to 418 | 340 to 432 |
| 337 to 429 | 338 to 421 | 336 to 419 | 340 to 433 |
| 337 to 430 | 338 to 422 | 336 to 420 | 340 to 434 |
| 337 to 431 | 338 to 423 | 336 to 421 | 340 to 435 |
| 337 to 432 | 338 to 424 | 336 to 422 | 340 to 436 |
| 337 to 433 | 338 to 425 | 336 to 423 | 340 to 437 |
| 337 to 434 | 338 to 426 | 336 to 424 | 341 to 403 |
| 337 to 435 | 338 to 427 | 336 to 425 | 341 to 404 |
| 337 to 436 | 338 to 428 | 336 to 426 | 341 to 405 |
| 337 to 437 | 338 to 429 | 336 to 427 | 341 to 406 |
| 337 to 438 | 338 to 430 | 336 to 428 | 341 to 407 |
| 337 to 439 | 338 to 431 | 336 to 429 | 341 to 408 |
| 337 to 440 | 338 to 432 | 336 to 430 | 341 to 409 |
| 337 to 441 | 338 to 433 | 336 to 431 | 341 to 410 |
| 337 to 442 | 338 to 434 | 336 to 432 | 341 to 411 |
| 337 to 443 | 338 to 435 | 336 to 433 | 341 to 412 |
| 337 to 444 | 338 to 436 | 336 to 434 | 341 to 413 |
| 337 to 445 | 338 to 437 | 336 to 435 | 341 to 414 |
| 337 to 446 | 338 to 438 | 336 to 436 | 341 to 415 |
| 337 to 447 | 338 to 439 | 336 to 437 | 341 to 416 |
| 338 to 406 | 338 to 440 | 336 to 438 | 341 to 417 |
| 338 to 407 | 339 to 404 | 337 to 402 | 341 to 418 |
| 338 to 408 | 339 to 405 | 337 to 403 | 341 to 419 |
| 338 to 409 | 339 to 406 | 337 to 404 | 341 to 420 |
| 338 to 410 | 339 to 407 | 337 to 405 | 341 to 421 |
| 338 to 411 | 339 to 408 | 337 to 406 | 341 to 422 |
| 338 to 412 | 339 to 409 | 337 to 407 | 341 to 423 |
| 338 to 413 | 339 to 410 | 337 to 408 | 341 to 424 |
| 338 to 414 | 339 to 411 | 337 to 409 | 341 to 425 |
| 338 to 415 | 339 to 412 | 337 to 410 | 341 to 426 |
| 338 to 416 | 339 to 413 | 337 to 411 | 341 to 427 |
| 338 to 417 | 339 to 414 | 337 to 412 | 341 to 428 |
| 338 to 418 | 339 to 415 | 337 to 413 | 341 to 429 |
| 338 to 419 | 339 to 416 | 337 to 414 | 341 to 430 |
| 338 to 420 | 339 to 417 | 337 to 415 | 341 to 431 |
| 338 to 421 | 339 to 418 | 337 to 416 | 341 to 432 |
| 338 to 422 | 339 to 419 | 337 to 417 | 341 to 433 |
| 338 to 423 | 339 to 420 | 337 to 418 | 341 to 434 |
| 338 to 424 | 339 to 421 | 337 to 419 | 341 to 435 |
| 338 to 425 | 339 to 422 | 337 to 420 | 341 to 436 |
| 338 to 426 | 339 to 423 | 337 to 421 | 341 to 437 |
| 338 to 427 | 339 to 424 | 337 to 422 | 342 to 403 |
| 338 to 428 | 339 to 425 | 337 to 423 | 342 to 404 |
| 338 to 429 | 339 to 426 | 337 to 424 | 342 to 405 |
| 338 to 430 | 339 to 427 | 337 to 425 | 342 to 406 |
| 338 to 431 | 339 to 428 | 337 to 426 | 342 to 407 |
| 338 to 432 | 339 to 429 | 337 to 427 | 342 to 408 |
| 338 to 433 | 339 to 430 | 337 to 428 | 342 to 409 |
| 338 to 434 | 339 to 431 | 337 to 429 | 342 to 410 |
| 338 to 435 | 339 to 432 | 337 to 430 | 342 to 411 |
| 338 to 436 | 339 to 433 | 337 to 431 | 342 to 412 |
| 338 to 437 | 339 to 434 | 337 to 432 | 342 to 413 |
| 338 to 438 | 339 to 435 | 337 to 433 | 342 to 414 |
| 338 to 439 | 339 to 436 | 337 to 434 | 342 to 415 |
| 338 to 440 | 339 to 437 | 337 to 435 | 342 to 416 |
| 338 to 441 | 339 to 438 | 337 to 436 | 342 to 417 |
| 338 to 442 | 339 to 439 | 337 to 437 | 342 to 418 |
| 338 to 443 | 339 to 440 | 337 to 438 | 342 to 419 |
| 338 to 444 | 340 to 404 | 338 to 402 | 342 to 420 |
| 338 to 445 | 340 to 405 | 338 to 403 | 342 to 421 |
| 338 to 446 | 340 to 406 | 338 to 404 | 342 to 422 |
| 338 to 447 | 340 to 407 | 338 to 405 | 342 to 423 |
| 339 to 406 | 340 to 408 | 338 to 406 | 342 to 424 |
| 339 to 407 | 340 to 409 | 338 to 407 | 342 to 425 |
| 339 to 408 | 340 to 410 | 338 to 408 | 342 to 426 |
| 339 to 409 | 340 to 411 | 338 to 409 | 342 to 427 |
| 339 to 410 | 340 to 412 | 338 to 410 | 342 to 428 |
| 339 to 411 | 340 to 413 | 338 to 411 | 342 to 429 |
| 339 to 412 | 340 to 414 | 338 to 412 | 342 to 430 |
| 339 to 413 | 340 to 415 | 338 to 413 | 342 to 431 |
| 339 to 414 | 340 to 416 | 338 to 414 | 342 to 432 |
| 339 to 415 | 340 to 417 | 338 to 415 | 342 to 433 |
| 339 to 416 | 340 to 418 | 338 to 416 | 342 to 434 |
| 339 to 417 | 340 to 419 | 338 to 417 | 342 to 435 |
| 339 to 418 | 340 to 420 | 338 to 418 | 342 to 436 |
| 339 to 419 | 340 to 421 | 338 to 419 | 342 to 437 |
| 339 to 420 | 340 to 422 | 338 to 420 | 343 to 403 |
| 339 to 421 | 340 to 423 | 338 to 421 | 343 to 404 |
| 339 to 422 | 340 to 424 | 338 to 422 | 343 to 405 |
| 339 to 423 | 340 to 425 | 338 to 423 | 343 to 406 |
| 339 to 424 | 340 to 426 | 338 to 424 | 343 to 407 |
| 339 to 425 | 340 to 427 | 338 to 425 | 343 to 408 |
| 339 to 426 | 340 to 428 | 338 to 426 | 343 to 409 |
| 339 to 427 | 340 to 429 | 338 to 427 | 343 to 410 |
| 339 to 428 | 340 to 430 | 338 to 428 | 343 to 411 |
| 339 to 429 | 340 to 431 | 338 to 429 | 343 to 412 |
| 339 to 430 | 340 to 432 | 338 to 430 | 343 to 413 |
| 339 to 431 | 340 to 433 | 338 to 431 | 343 to 414 |
| 339 to 432 | 340 to 434 | 338 to 432 | 343 to 415 |
| 339 to 433 | 340 to 435 | 338 to 433 | 343 to 416 |
| 339 to 434 | 340 to 436 | 338 to 434 | 343 to 417 |
| 339 to 435 | 340 to 437 | 338 to 435 | 343 to 418 |
| 339 to 436 | 340 to 438 | 338 to 436 | 343 to 419 |
| 339 to 437 | 340 to 439 | 338 to 437 | 343 to 420 |
| 339 to 438 | 340 to 440 | 338 to 438 | 343 to 421 |
| 339 to 439 | 341 to 404 | 339 to 402 | 343 to 422 |
| 339 to 440 | 341 to 405 | 339 to 403 | 343 to 423 |
| 339 to 441 | 341 to 406 | 339 to 404 | 343 to 424 |
| 339 to 442 | 341 to 407 | 339 to 405 | 343 to 425 |
| 339 to 443 | 341 to 408 | 339 to 406 | 343 to 426 |
| 339 to 444 | 341 to 409 | 339 to 407 | 343 to 427 |
| 339 to 445 | 341 to 410 | 339 to 408 | 343 to 428 |
| 339 to 446 | 341 to 411 | 339 to 409 | 343 to 429 |
| 339 to 447 | 341 to 412 | 339 to 410 | 343 to 430 |
| 340 to 406 | 341 to 413 | 339 to 411 | 343 to 431 |
| 340 to 407 | 341 to 414 | 339 to 412 | 343 to 432 |
| 340 to 408 | 341 to 415 | 339 to 413 | 343 to 433 |
| 340 to 409 | 341 to 416 | 339 to 414 | 343 to 434 |
| 340 to 410 | 341 to 417 | 339 to 415 | 343 to 435 |
| 340 to 411 | 341 to 418 | 339 to 416 | 343 to 436 |
| 340 to 412 | 341 to 419 | 339 to 417 | 343 to 437 |
| 340 to 413 | 341 to 420 | 339 to 418 | |
| 340 to 414 | 341 to 421 | 339 to 419 | |
| 340 to 415 | 341 to 422 | 339 to 420 | |
| 340 to 416 | 341 to 423 | 339 to 421 | |
| 340 to 417 | 341 to 424 | 339 to 422 | |
| 340 to 418 | 341 to 425 | 339 to 423 | |
| 340 to 419 | 341 to 426 | 339 to 424 | |
| 340 to 420 | 341 to 427 | 339 to 425 | |
| 340 to 421 | 341 to 428 | 339 to 426 | |
| 340 to 422 | 341 to 429 | 339 to 427 | |
| 340 to 423 | 341 to 430 | 339 to 428 | |
| 340 to 424 | 341 to 431 | 339 to 429 | |
| 340 to 425 | 341 to 432 | 339 to 430 | |
| 340 to 426 | 341 to 433 | 339 to 431 | |
| 340 to 427 | 341 to 434 | 339 to 432 | |
| 340 to 428 | 341 to 435 | 339 to 433 | |
| 340 to 429 | 341 to 436 | 339 to 434 | |
| 340 to 430 | 341 to 437 | 339 to 435 | |
| 340 to 431 | 341 to 438 | 339 to 436 | |
| 340 to 432 | 341 to 439 | 339 to 437 | |
| 340 to 433 | 341 to 440 | 339 to 438 | |
| 340 to 434 | 342 to 404 | 340 to 402 | |
| 340 to 435 | 342 to 405 | 340 to 403 | |
| 340 to 436 | 342 to 406 | 340 to 404 | |
| 340 to 437 | 342 to 407 | 340 to 405 | |
| 340 to 438 | 342 to 408 | 340 to 406 | |
| 340 to 439 | 342 to 409 | 340 to 407 | |
| 340 to 440 | 342 to 410 | 340 to 408 | |
| 340 to 441 | 342 to 411 | 340 to 409 | |
| 340 to 442 | 342 to 412 | 340 to 410 | |
| 340 to 443 | 342 to 413 | 340 to 411 | |
| 340 to 444 | 342 to 414 | 340 to 412 | |
| 340 to 445 | 342 to 415 | 340 to 413 | |
| 340 to 446 | 342 to 416 | 340 to 414 | |
| 340 to 447 | 342 to 417 | 340 to 415 | |
| 341 to 406 | 342 to 418 | 340 to 416 | |
| 341 to 407 | 342 to 419 | 340 to 417 | |
| 341 to 408 | 342 to 420 | 340 to 418 | |
| 341 to 409 | 342 to 421 | 340 to 419 | |
| 341 to 410 | 342 to 422 | 340 to 420 | |
| 341 to 411 | 342 to 423 | 340 to 421 | |
| 341 to 412 | 342 to 424 | 340 to 422 | |
| 341 to 413 | 342 to 425 | 340 to 423 | |
| 341 to 414 | 342 to 426 | 340 to 424 | |
| 341 to 415 | 342 to 427 | 340 to 425 | |
| 341 to 416 | 342 to 428 | 340 to 426 | |
| 341 to 417 | 342 to 429 | 340 to 427 | |
| 341 to 418 | 342 to 430 | 340 to 428 | |
| 341 to 419 | 342 to 431 | 340 to 429 | |
| 341 to 420 | 342 to 432 | 340 to 430 | |
| 341 to 421 | 342 to 433 | 340 to 431 | |
| 341 to 422 | 342 to 434 | 340 to 432 | |
| 341 to 423 | 342 to 435 | 340 to 433 | |
| 341 to 424 | 342 to 436 | 340 to 434 | |
| 341 to 425 | 342 to 437 | 340 to 435 | |
| 341 to 426 | 342 to 438 | 340 to 436 | |
| 341 to 427 | 342 to 439 | 340 to 437 | |
| 341 to 428 | 342 to 440 | 340 to 438 | |
| 341 to 429 | | | |
| 341 to 430 | | | |
| 341 to 431 | | | |
| 341 to 432 | | | |
| 341 to 433 | | | |
| 341 to 434 | | | |
| 341 to 435 | | | |
| 341 to 436 | | | |
| 341 to 437 | | | |
| 341 to 438 | | | |
| 341 to 439 | | | |
| 341 to 440 | | | |
| 341 to 441 | | | |
| 341 to 442 | | | |
| 341 to 443 | | | |
| 341 to 444 | | | |
| 341 to 445 | | | |
| 341 to 446 | | | |
| 341 to 447 | | | |
| 342 to 406 | | | |
| 342 to 407 | | | |
| 342 to 408 | | | |
| 342 to 409 | | | |
| 342 to 410 | | | |
| 342 to 411 | | | |
| 342 to 412 | | | |
| 342 to 413 | | | |
| 342 to 414 | | | |
| 342 to 415 | | | |
| 342 to 416 | | | |
| 342 to 417 | | | |
| 342 to 418 | | | |
| 342 to 419 | | | |
| 342 to 420 | | | |
| 342 to 421 | | | |
| 342 to 422 | | | |
| 342 to 423 | | | |
| 342 to 424 | | | |
| 342 to 425 | | | |
| 342 to 426 | | | |
| 342 to 427 | | | |
| 342 to 428 | | | |
| 342 to 429 | | | |
| 342 to 430 | | | |
| 342 to 431 | | | |
| 342 to 432 | | | |
| 342 to 433 | | | |
| 342 to 434 | | | |
| 342 to 435 | | | |
| 342 to 436 | | | |
| 342 to 437 | | | |
| 342 to 438 | | | |
| 342 to 439 | | | |
| 342 to 440 | | | |
| 342 to 441 | | | |
| 342 to 442 | | | |
| 342 to 443 | | | |
| 342 to 444 | | | |
| 342 to 445 | | | |
| 342 to 446 | | | |
| 342 to 447 | | | |
| 343 to 406 | | | |
| 343 to 407 | | | |
| 343 to 408 | | | |
| 343 to 409 | | | |
| 343 to 410 | | | |
| 343 to 411 | | | |
| 343 to 412 | | | |
| 343 to 413 | | | |
| 343 to 414 | | | |
| 343 to 415 | | | |
| 343 to 416 | | | |
| 343 to 417 | | | |
| 343 to 418 | | | |
| 343 to 419 | | | |
| 343 to 420 | | | |
| 343 to 421 | | | |
| 343 to 422 | | | |
| 343 to 423 | | | |
| 343 to 424 | | | |
| 343 to 425 | | | |
| 343 to 426 | | | |
| 343 to 427 | | | |
| 343 to 428 | | | |
| 343 to 429 | | | |
| 343 to 430 | | | |
| 343 to 431 | | | |
| 343 to 432 | | | |
| 343 to 433 | | | |
| 343 to 434 | | | |
| 343 to 435 | | | |
| 343 to 436 | | | |
| 343 to 437 | | | |
| 343 to 438 | | | |
| 343 to 439 | | | |
| 343 to 440 | | | |
| 343 to 441 | | | |
| 343 to 442 | | | |
| 343 to 443 | | | |
| 343 to 444 | | | |
| 343 to 445 | | | |
| 343 to 446 | | | |
| 343 to 447 | | | |
| 344 to 406 | | | |
| 344 to 407 | | | |
| 344 to 408 | | | |
| 344 to 409 | | | |
| 344 to 410 | | | |
| 344 to 411 | | | |
| 344 to 412 | | | |
| 344 to 413 | | | |
| 344 to 414 | | | |
| 344 to 415 | | | |
| 344 to 416 | | | |
| 344 to 417 | | | |
| 344 to 418 | | | |
| 344 to 419 | | | |
| 344 to 420 | | | |
| 344 to 421 | | | |
| 344 to 422 | | | |
| 344 to 423 | | | |
| 344 to 424 | | | |
| 344 to 425 | | | |
| 344 to 426 | | | |
| 344 to 427 | | | |
| 344 to 428 | | | |
| 344 to 429 | | | |
| 344 to 430 | | | |
| 344 to 431 | | | |
| 344 to 432 | | | |
| 344 to 433 | | | |
| 344 to 434 | | | |
| 344 to 435 | | | |
| 344 to 436 | | | |
| 344 to 437 | | | |
| 344 to 438 | | | |
| 344 to 439 | | | |
| 344 to 440 | | | |
| 344 to 441 | | | |
| 344 to 442 | | | |
| 344 to 443 | | | |
| 344 to 444 | | | |
| 344 to 445 | | | |
| 344 to 446 | | | |
| 344 to 447 | | | |

TABLE 2
Percentage inhibition of sFLT-1 binding to a monoclonal antibody (2B2)
after pre-incubation with 50 μg/ml Domain 4 constructs 0, 1, and 2 and
25 μg/ml Domain 4 construct 3 as determined by BIAcore analysis.

| Sample | % inhibition of sFLT-1 |
|---|---|
| Domain 4 construct 0 | 5.85 % |
| Domain 4 construct 1 | 64.66 % |
| Domain 4 construct 2 | 73.35 % |
| Domain 4 construct 3 | 44.87 % |

1. A protein selected from the group consisting of the amino acid sequence of the fourth Ig-like domain of a VEGF receptor, and a variant of said protein that retains the ability to bind to a VEGF

2. A protein according to claim 1, wherein said VEGF receptor belongs to either the FLT receptor family or the KDR/FLK receptor family.

3. A protein according to claim 2, wherein said VEGF receptor is the FLT-1 receptor, the KDR/FLK receptor or the FLT-4 receptor.

4. A protein according to claim 1, wherein said VEGF receptor is a human VEGF receptor.

5. A protein according to claim 1, wherein said protein binds to the fourth Ig-like domain of a VEGF receptor.

6. A protein according to claim 1 that binds to the fourth Ig-like domain of a VEGF receptor with a dissociation constant of 2 μM or less, preferably, 0.2 μM or less, more preferably 2 nM or less, even more preferably, 20 pm or less.

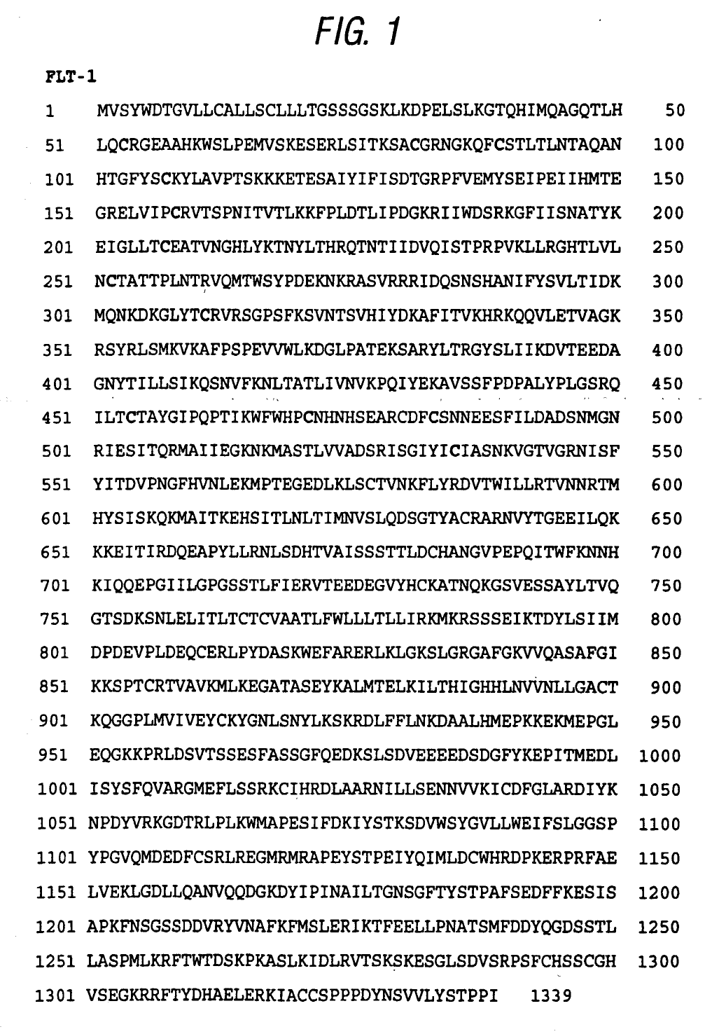

7. A protein according to claim 1, wherein said fourth Ig-like domain comprises at least residues 344-406 of the full length FLT-1 sequence given in FIG. 1, but no more than residues 316-447 of this sequence.

8. A protein according to claim 7 which consists of residues 338-440, 330-429, 330-440 or 338-429 of the full length FLT-1 sequence or a variant of this sequence containing one or more amino acid substitutions that do not decrease the binding affinity of the protein for the fourth Ig-like domain of a VEGF receptor.

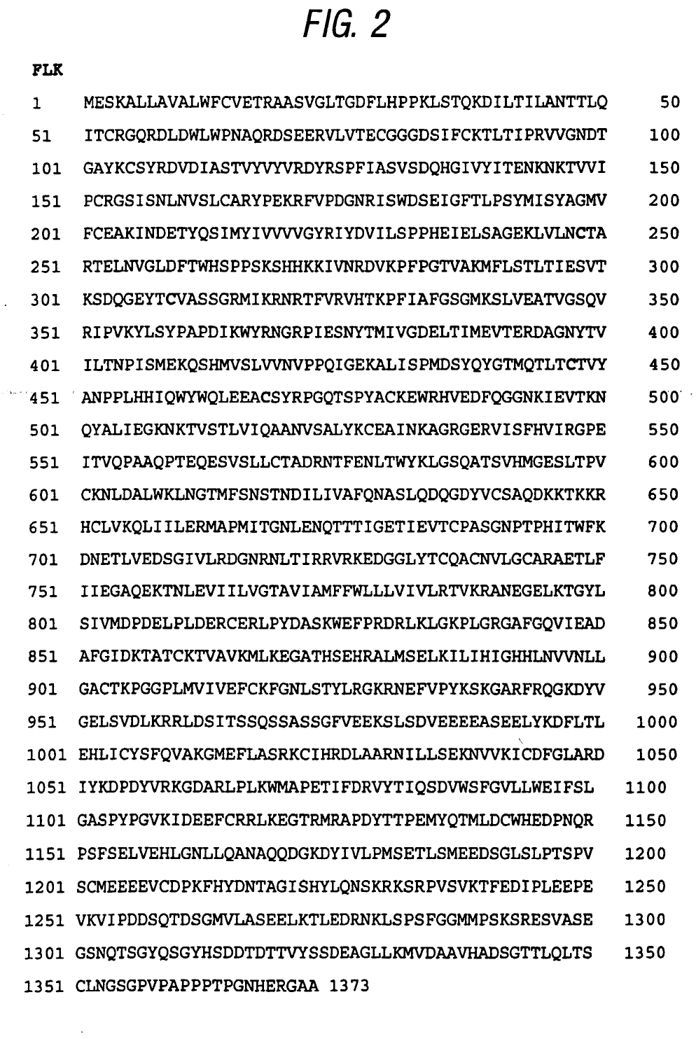

9. A protein according to claim 1, wherein said fourth Ig-like domain comprises at least residues 342-404 of the full length FLK sequence given in FIG. 2, but no more than residues 314-440 of this sequence.

10. A protein according to claim 9 which consists of residues 335-435, 328-424, 328-435 or 335-424 of the full length FLK sequence or a variant of this sequence containing one or more amino acid substitutions that do not decrease the binding affinity of the protein for the fourth Ig-like domain of a VEGF receptor.

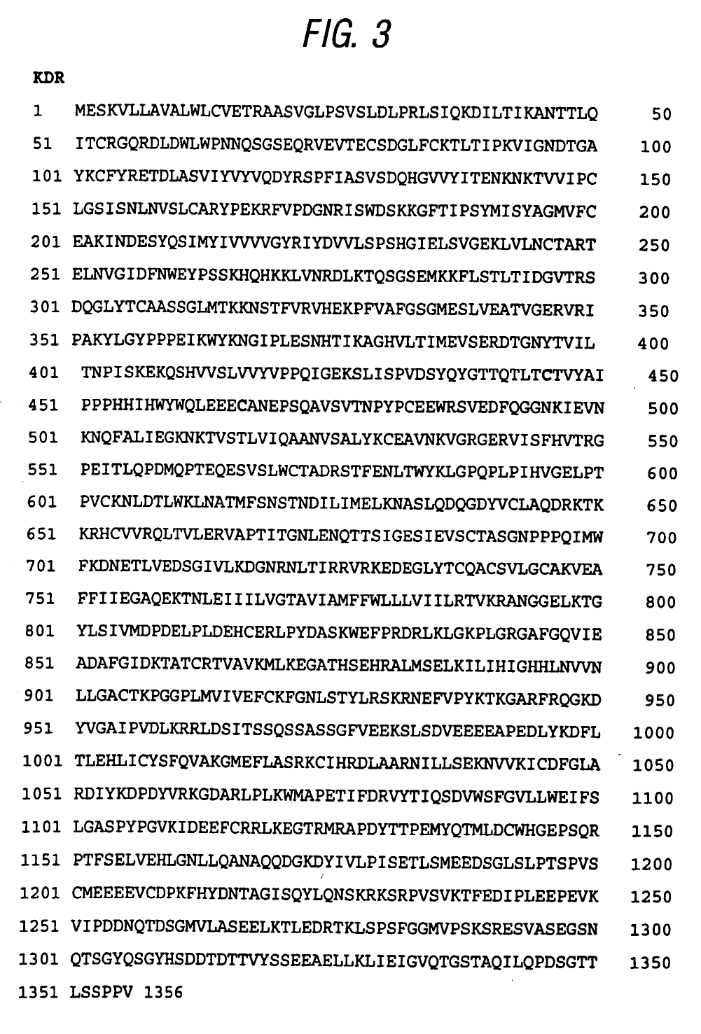

11. A protein according to claim 1, wherein said fourth Ig-like domain comprises at least residues 340 to 402 of the full length KDR sequence given in FIG. 3, but no more than residues 312-438 of this sequence.

12. A protein according to claim 11 which consists of residues 333-433, 326-422, 326-433 or 333-422 of the full length KDR sequence or a variant of this sequence containing one or more amino acid substitutions that do not decrease the binding affinity of the protein for the fourth Ig-like domain of a VEGF receptor.

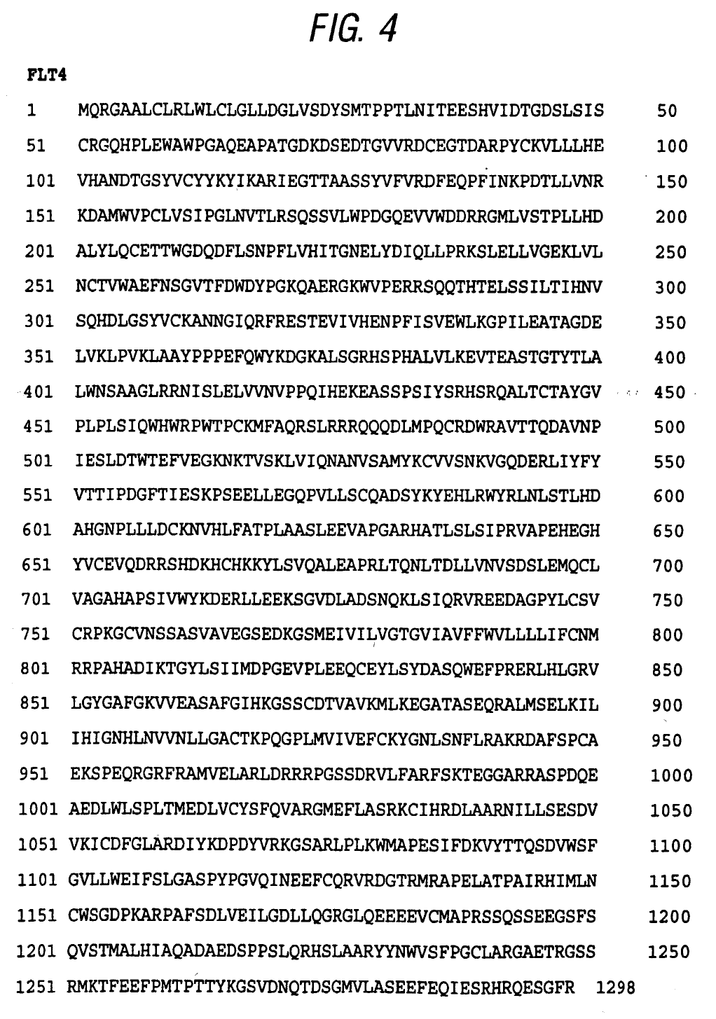

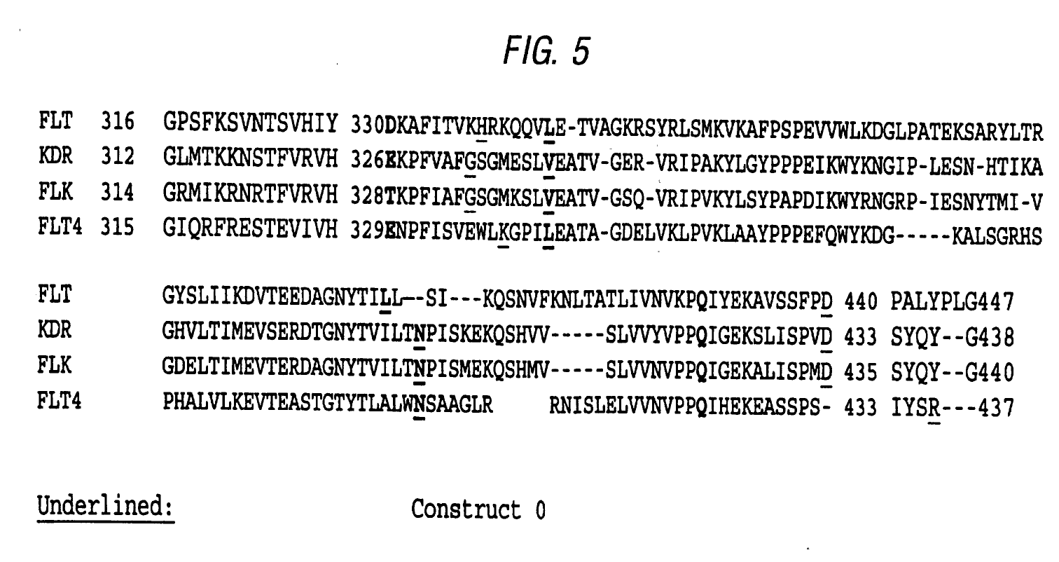

13. A protein according to claim 1, wherein said fourth Ig-like domain comprises residues 343-403 of the full length FLT-4 sequence given in FIG. 4, but no more than residues 315 to 437 of this sequence.

14. A protein according to claim 13 which consists of residues 339-437, 329-423, 329-437 or 339-423 of the full length FLT-4 sequence or a variant of this sequence containing one or more amino acid substitutions that do not decrease the binding affinity of the protein for the fourth Ig-like domain of a VEGF receptor.

15. A protein according to claim 1 that has been genetically or chemically fused to one or more peptides or polypeptides.

16. A protein according to claim 15, which comprises repeated amino acid sequences of the fourth Ig-like domain or a VEGF receptor.

17. A protein according to claim 15 or claim 16, fused to a label.

18. A protein according to claim 1, for use as a pharmaceutical.

19. A functional equivalent of a protein according to claim 1 that binds to the fourth Ig-like domain of a VEGF receptor.

20. A functional equivalent according to claim 19, which is an antiidiotypic antibody, a peptide, an oligopeptide, a peptidomimetic compound or a drug molecule, such as a small natural or synthetic organic molecule.

21. A nucleic acid encoding a protein according to claim 1.

22. A vector comprising a nucleic acid according to claim 21.

23. A host cell comprising a vector according to claim 22.

24. A pharmaceutical composition for the treatment of a disorder whose pathology is dependent upon a VEGF family-mediated pathway comprising a protein according to claim 1 or a functional equivalent according to claim 19 or 20, in association with a suitable pharmaceutical excipient.

25. A method of treating a patient suffering from a disorder whose pathology is dependent upon a VEGF-mediated pathway comprising administering to a patient a therapeutically-effective amount of a protein according to claim 1, a functional equivalent according to claim 19, a nucleic acid according to claim 21, or a pharmaceutical composition according to claim 24.

26. A method according to claim 25, wherein said disorder is an inflammation, psoriasis, rheumatoid arthritis, hemangiomas, leiomyomas, diabetic retinopathy, angiofibromas, endometriosis. macular degeneration, retinal neovascularisation or cancer.

27. A transgenic animal that has been transformed by a nucleic acid molecule according to claim 21 or a vector according to claim 22.

28. A method for inhibiting the dimerisation of a VEGF receptor, comprising bringing the receptor into contact with a protein, or functional equivalent according to claim 1.
